SEQUENCE LISTING (91 sequences)

SEQ ID NO: 1 - DNA, 1004 bp, Homo sapiens
gcctcgctcgggcgcccagtggtcctgccgcctggtctcacctcgccatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtgaggctgcacccacccaggagtgtggccacgtgggcaaacaggcagttggccagagagcctggtgctgctgctgcaggggtgcaggcagaagcggggagctatgcccagtcagtgccagcccctc

SEQ ID NO: 2 - DNA, 938 bp, Mus musculus
gcctcctggcccttcagctgtggtctttcccgttttctgactttgcggtgacactggggacttccttagacctctctggagacgctttcggttctgcagagattcccaggggtattgtgggtggggtggggtaacaatagtgtccctgtggcgctcccagtccctatagtaatccttcacccctctgctatcttgcaatcaggagagtccttagccctgctataggtggcttttgaggtcctggatgcgaggagggggactggggggtgggtcgggtaatgtaagaaaagggctccttttgggaccctggctcctccagccaccttggtgcccatcccttaaactcttggggacaatcagactcctgggaaggtcctggggaaatccctgctcagtgactagccataggcccaccgcgattggtgcccgaagaccccgccctcttcctgggcgggactcctagcagggactttggagtgacttgtggcttcagcaggagccctgtgatttggctcttctgatctcgccctgcgatggtgtctttgcctcggctgtgcgcgctatggggctgcttgttgacagcggtgagtggcttgtgttctaacctccaagggagttagggcttagagagtgagagatggaaagaggaaagaggagacaagactttggagatgagagatcttcctactggaagcggcggttagtaggatgggcaagatctctcgcgtcttgacacacacacacacacacacaaatgaggtgggctgctcctctttccttccagaaggtcggggttctgttccacgaagcccacagggaaccttagggagggcattcctccacagcggtgcctggacagctttgtctgacccaagccttgctccggagctgactgcagagactggaaagggttagcagacaggaagcctggctggggg

SEQ ID NO: 3 - DNA, 547 bp, Rattus sp.
  modified_base (334): a, c, g, t, unknown or other
tgggacccctgtgatctggctgctctgatctcgctctgcaatgctgcctttgcctcagctgtgcgcgctctggggctgcttgttgacagcggtccatctaggacagtgtgttacgtgcagtgacaaacagtacctccaaggtggcgagtgctgcgatttgtgccagccgggaaaccgactagttagccactgcacagctcttgagaagacccaatgccaaccgtgcgactcaggcgaattctcagctcactggaacagggagatccgctgccaccagcaccgacactgcgaactcaatcaagggcttcaggttaagaaggagggcaccgcggtntcagacactgtttgtacctgcaaggaagggcagcactgcgccagcaaggagtgcgagacgtgcgctcagcacaggccctgtggccctggctttggagtcgtgcagatggccactgagactactgataccgtctgccaaccctgcccggtcggattcttctccaatgggtcatcactttttgaaaagtgtcatccatggacaagctgtgaagat

SEQ ID NO: 4 - DNA, 1284 bp, Sus sp.
gcctcgccatggttcgtctgcctctgaagtgtctcctctggggctgctttttgaccgccgtccacccagaaccacccacttcatgcaaagaaaaccaatacccaacaaacagccggtgctgtaatttgtgcccgccaggacagaaactggtgaaccactgcacagaggtcactgaaacagaatgccttccttgcagttccagcgaattcctagccacctggaatagagagaaacactgtcatcagcacaaatactgcgaccccaacctaggtctccaggtccagagggagggcacctcgaaaacagacaccacttgtgtgtgcagtgaaggccatcactgtaccaacagcgcctgtgaaagttgcaccttgcacagcttgtgcttccctggcctcggggtcaagcagatggcgacagaggtttctgacactatctgtgaaccctgcccagttggcttcttctccaatgtatcatctgcttcagaaaagtgtcagccttggacaagctgcgagagcaaaggcctggtggaacaacgtgcggggactaacaagaccgatgttgtctgtggtttccagagtcggatgagagccctggtggttatccccatcacgctggggatcctgtttgccgtcctgttggtatttctctgtatcagaaaggtgaccaaggagcaggagactaaggccctgcaccctaagactgaaaggcaggatcccgtggagacgattgatctggaggattttcccgactccaccgctccggtgcaggagaccttacattggtgccagcccgtcacccaggaggatggcaaagagagccgcatctccgtgcaggagcgagagtgaggctgtgcgtggccaggagcgtggaggcacgggcacaggggcatgtgactggagagcccggggcggctgctgctgctgtggcggtggtgagagggtggtgctgggcacagccccttctgcctgcacccctgcagtccagatacagtccacctcgaggagcttctcaccccagccctggagcccattcaatctcagtttgcttttaaagatggagacaaaactttggggagtcacagccacagtaataaccaccagagcttccaacccagaggttcagtacctgcagatgcaagggatggcgtctaggagcccaggaggcatatacatgactgtccaccactgcattgttcgtgacagtgagtgactggaaactgcttaactgtccatcaacaggggactggctaaataaaattgtaacatgtttatgcaaaaaaaaaaa

SEQ ID NO: 5 - DNA, 18 bp, Artificial Sequence
  Description of Artificial Sequence Synthetic oligonucleotide
ccaggcggcaggaccact

SEQ ID NO: 6 - DNA, 18 bp, Artificial Sequence
  Description of Artificial Sequence Synthetic oligonucleotide
gaccaggcggcaggacca

SEQ ID NO: 7 - DNA, 18 bp, Artificial Sequence
  Description of Artificial Sequence Synthetic oligonucleotide
aggtgagaccaggcggca

SEQ ID NO: 8 - DNA, 18 bp, Artificial Sequence
  Description of Artificial Sequence Synthetic oligonucleotide
gcagaggcagacgaacca

SEQ ID NO: 9 - DNA, 18 bp, Artificial Sequence
  Description of Artificial Sequence Synthetic oligonucleotide
gcaagcagccccagagga

SEQ ID NO: 10 - DNA, 18 bp, Artificial Sequence
  Description of Artificial Sequence Synthetic oligonucleotide
ggtcagcaagcagcccca

SEQ ID NO: 11 - DNA, 18 bp, Artificial Sequence
  Description of Artificial Sequence Synthetic oligonucleotide
gacagcggtcagcaagca

SEQ ID NO: 12 - DNA, 18 bp, Artificial Sequence
  Description of Artificial Sequence Synthetic oligonucleotide
gatggacagcggtcagca

SEQ ID NO: 13 - DNA, 18 bp, Artificial Sequence
  Description of Artificial Sequence Synthetic oligonucleotide
tctggatggacagcggtc

SEQ ID NO: 14 - DNA, 18 bp, Artificial Sequence
  Description of Artificial Sequence Synthetic oligonucleotide
ggtggttctggatggaca

SEQ ID NO: 15 - DNA, 18 bp, Artificial Sequence
  Description of Artificial Sequence Synthetic oligonucleotide
gtgggtggttctggatgg

SEQ ID NO: 16 - DNA, 18 bp, Artificial Sequence
  Description of Artificial Sequence Synthetic oligonucleotide
gcagtgggtggttctgga

SEQ ID NO: 17 - DNA, 18 bp, Artificial Sequence
  Description of Artificial Sequence Synthetic oligonucleotide
ctggcacaaagaacagca

SEQ ID NO: 18 - DNA, 18 bp, Artificial Sequence
  Description of Artificial Sequence Synthetic oligonucleotide
gtgcagtcactcaccagt

SEQ ID NO: 19 - DNA, 18 bp, Artificial Sequence
  Description of Artificial Sequence Synthetic oligonucleotide
attccgtttcagtgaact

SEQ ID NO: 20 - DNA, 18 bp, Artificial Sequence
  Description of Artificial Sequence Synthetic oligonucleotide
ttcaccgcaaggaaggca

SEQ ID NO: 21 - DNA, 18 bp, Artificial Sequence
  Description of Artificial Sequence Synthetic oligonucleotide
ctctgttccaggtgtcta

SEQ ID NO: 22 - DNA, 18 bp, Artificial Sequence
  Description of Artificial Sequence Synthetic oligonucleotide
ctggtggcagtgtgtctc

SEQ ID NO: 23 - DNA, 18 bp, Artificial Sequence
  Description of Artificial Sequence Synthetic oligonucleotide
ggtgcccttctgctggac

SEQ ID NO: 24 - DNA, 18 bp, Artificial Sequence
  Description of Artificial Sequence Synthetic oligonucleotide
ctgaggtgcccttctgct

SEQ ID NO: 25 - DNA, 18 bp, Artificial Sequence
  Description of Artificial Sequence Synthetic oligonucleotide
gtgtctgtttctgaggtg

SEQ ID NO: 26 - DNA, 18 bp, Artificial Sequence
  Description of Artificial Sequence Synthetic oligonucleotide
acaggtgcagatggtgtc

SEQ ID NO: 27 - DNA, 18 bp, Artificial Sequence
  Description of Artificial Sequence Synthetic oligonucleotide
gtgccagccttcttcaca

SEQ ID NO: 28 - DNA, 18 bp, Artificial Sequence
  Description of Artificial Sequence Synthetic oligonucleotide
tgcaggacacagctctca

SEQ ID NO: 29 - DNA, 18 bp, Artificial Sequence
  Description of Artificial Sequence Synthetic oligonucleotide
gagcggtgcaggacacag

SEQ ID NO: 30 - DNA, 18 bp, Artificial Sequence
  Description of Artificial Sequence Synthetic oligonucleotide
aatctgcttgaccccaaa

SEQ ID NO: 31 - DNA, 18 bp, Artificial Sequence
  Description of Artificial Sequence Synthetic oligonucleotide
gctcgcagatggtatcag

SEQ ID NO: 32 - DNA, 18 bp, Artificial Sequence
  Description of Artificial Sequence Synthetic oligonucleotide
gcagggctcgcagatggt

SEQ ID NO: 33 - DNA, 18 bp, Artificial Sequence
  Description of Artificial Sequence Synthetic oligonucleotide
gactgggcagggctcgca

SEQ ID NO: 34 - DNA, 18 bp, Artificial Sequence
  Description of Artificial Sequence Synthetic oligonucleotide
gcagatgacacattggag

SEQ ID NO: 35 - DNA, 18 bp, Artificial Sequence
  Description of Artificial Sequence Synthetic oligonucleotide
tcgaaagcagatgacaca

SEQ ID NO: 36 - DNA, 18 bp, Artificial Sequence
  Description of Artificial Sequence Synthetic oligonucleotide
gtccaagggtgacatttt

SEQ ID NO: 37 - DNA, 18 bp, Artificial Sequence
  Description of Artificial Sequence Synthetic oligonucleotide
caggtctttggtctcaca

SEQ ID NO: 38 - DNA, 18 bp, Artificial Sequence
  Description of Artificial Sequence Synthetic oligonucleotide
ctgttgcacaaccaggtc

SEQ ID NO: 39 - DNA, 18 bp, Artificial Sequence
  Description of Artificial Sequence Synthetic oligonucleotide
gtttgtgcctgcctgttg

SEQ ID NO: 40 - DNA, 18 bp, Artificial Sequence
  Description of Artificial Sequence Synthetic oligonucleotide
gtcttgtttgtgcctgcc

SEQ ID NO: 41 - DNA, 18 bp, Artificial Sequence
  Description of Artificial Sequence Synthetic oligonucleotide
caccaccagggctctcag

SEQ ID NO: 42 - DNA, 18 bp, Artificial Sequence
  Description of Artificial Sequence Synthetic oligonucleotide
gggatcaccaccagggct

SEQ ID NO: 43 - DNA, 18 bp, Artificial Sequence
  Description of Artificial Sequence Synthetic oligonucleotide
gtcgggaaaattgatctc

SEQ ID NO: 44 - DNA, 18 bp, Artificial Sequence
  Description of Artificial Sequence Synthetic oligonucleotide
ggagccaggaagatcgtc

SEQ ID NO: 45 - DNA, 18 bp, Artificial Sequence
  Description of Artificial Sequence Synthetic oligonucleotide
tggagccaggaagatcgt

SEQ ID NO: 46 - DNA, 18 bp, Artificial Sequence
  Description of Artificial Sequence Synthetic oligonucleotide
tggcatccatgtaaagtc

SEQ ID NO: 47 - DNA, 18 bp, Artificial Sequence
  Description of Artificial Sequence Synthetic oligonucleotide
ggtgcagcctcactgtct

SEQ ID NO: 48 - DNA, 18 bp, Artificial Sequence
  Description of Artificial Sequence Synthetic oligonucleotide
aactgcctgtttgcccac

SEQ ID NO: 49 - RNA, 21 nt, Artificial Sequence
  Description of Artificial Sequence Synthetic siRNA sequence
gcgaauuccuagacaccuguu

SEQ ID NO: 50 - RNA, 21 nt, Artificial Sequence
  Description of Artificial Sequence Synthetic siRNA sequence
cuggugagugacugcacaguu

SEQ ID NO: 51 - RNA, 21 nt, Artificial Sequence
  Description of Artificial Sequence Synthetic siRNA sequence
uacugcgaccccaaccuaguu

SEQ ID NO: 52 - DNA, 14 bp, Artificial Sequence
  Description of Artificial Sequence Synthetic oligonucleotide
agacaccatcgcag

SEQ ID NO: 53 - DNA, 15 bp, Artificial Sequence
  Description of Artificial Sequence Synthetic oligonucleotide
gcgagatcagaagag

SEQ ID NO: 54 - DNA, 15 bp, Artificial Sequence
  Description of Artificial Sequence Synthetic oligonucleotide
cgctgtcaacaagca

SEQ ID NO: 55 - DNA, 15 bp, Artificial Sequence
  Description of Artificial Sequence Synthetic oligonucleotide
ctgccctagatggac

SEQ ID NO: 56 - DNA, 15 bp, Artificial Sequence
  Description of Artificial Sequence Synthetic oligonucleotide
ctggctggcacaaat

SEQ ID NO: 57 - DNA, 15 bp, Artificial Sequence
  Description of Artificial Sequence Synthetic oligonucleotide
cttgtccagggataa

SEQ ID NO: 58 - DNA, 15 bp, Artificial Sequence
  Description of Artificial Sequence Synthetic oligonucleotide
cacagatgacattag

SEQ ID NO: 59 - DNA, 15 bp, Artificial Sequence
  Description of Artificial Sequence Synthetic oligonucleotide
tgatatagagaaaca

SEQ ID NO: 60 - DNA, 15 bp, Artificial Sequence
  Description of Artificial Sequence Synthetic oligonucleotide
ctcattatcctttgg

SEQ ID NO: 61 - DNA, 13 bp, Artificial Sequence
  Description of Artificial Sequence Synthetic oligonucleotide
ggttcagaccagg

SEQ ID NO: 62 - DNA, 15 bp, Artificial Sequence
  Description of Artificial Sequence Synthetic oligonucleotide
tttatttagccagta

SEQ ID NO: 63 - DNA, 15 bp, Artificial Sequence
  Description of Artificial Sequence Synthetic oligonucleotide
agccccacgcactgg

SEQ ID NO: 64 - DNA, 18 bp, Artificial Sequence
  Description of Artificial Sequence Synthetic oligonucleotide
tctcactcctatcccagt

SEQ ID NO: 65 - DNA, 15 bp, Artificial Sequence
  Description of Artificial Sequence Synthetic oligonucleotide
attagtctgactcgt

SEQ ID NO: 66 - DNA, 15 bp, Artificial Sequence
  Description of Artificial Sequence Synthetic oligonucleotide
acattagtctgactc

SEQ ID NO: 67 - DNA, 15 bp, Artificial Sequence
  Description of Artificial Sequence Synthetic oligonucleotide
cagatgacattagtc

SEQ ID NO: 68 - DNA, 15 bp, Artificial Sequence
  Description of Artificial Sequence Synthetic oligonucleotide
ctggactcaccacag

SEQ ID NO: 69 - DNA, 15 bp, Artificial Sequence
  Description of Artificial Sequence Synthetic oligonucleotide
ggactcaccacagat

SEQ ID NO: 70 - DNA, 15 bp, Artificial Sequence
  Description of Artificial Sequence Synthetic oligonucleotide
actcaccacagatga

SEQ ID NO: 71 - DNA, 15 bp, Artificial Sequence
  Description of Artificial Sequence Synthetic oligonucleotide
tcaccacagatgaca

SEQ ID NO: 72 - DNA, 15 bp, Artificial Sequence
  Description of Artificial Sequence Synthetic oligonucleotide
accacagatgacatt

SEQ ID NO: 73 - DNA, 12 bp, Artificial Sequence
  Description of Artificial Sequence Synthetic oligonucleotide
agatgacattag

SEQ ID NO: 74 - DNA, 13 bp, Artificial Sequence
  Description of Artificial Sequence Synthetic oligonucleotide
cagatgacattag

SEQ ID NO: 75 - DNA, 14 bp, Artificial Sequence
  Description of Artificial Sequence Synthetic oligonucleotide
acagatgacattag

SEQ ID NO: 76 - DNA, 16 bp, Artificial Sequence
  Description of Artificial Sequence Synthetic oligonucleotide
ccacagatgacattag

SEQ ID NO: 77 - DNA, 17 bp, Artificial Sequence
  Description of Artificial Sequence Synthetic oligonucleotide
accacagatgacattag

SEQ ID NO: 78 - DNA, 18 bp, Artificial Sequence
  Description of Artificial Sequence Synthetic oligonucleotide
caccacagatgacattag

SEQ ID NO: 79 - DNA, 19 bp, Artificial Sequence
  Description of Artificial Sequence Synthetic oligonucleotide
tcaccacagatgacattag

SEQ ID NO: 80 - DNA, 20 bp, Artificial Sequence
  Description of Artificial Sequence Synthetic oligonucleotide
ctcaccacagatgacattag

SEQ ID NO: 81 - DNA, 19 bp, Artificial Sequence
  Description of Artificial Sequence Synthetic oligonucleotide
accgctgtcaacaagcagc

SEQ ID NO: 82 - DNA, 18 bp, Artificial Sequence
  Description of Artificial Sequence Synthetic oligonucleotide
tcctagatggaccgctgt

SEQ ID NO: 83 - DNA, 16 bp, Artificial Sequence
  Description of Artificial Sequence Synthetic oligonucleotide
taacacactgtcctag

SEQ ID NO: 84 - DNA, 18 bp, Artificial Sequence
  Description of Artificial Sequence Synthetic oligonucleotide
gctgatgacagtgtttct

SEQ ID NO: 85 - DNA, 18 bp, Artificial Sequence
  Description of Artificial Sequence Synthetic oligonucleotide
gcctcactctcgctcctg

SEQ ID NO: 86 - DNA, 18 bp, Artificial Sequence
  Description of Artificial Sequence Synthetic oligonucleotide
ggactgtatctggactgc

SEQ ID NO: 87 - DNA, 18 bp, Artificial Sequence
  Description of Artificial Sequence Synthetic oligonucleotide
gtggacagtcatgtatat

SEQ ID NO: 88 - DNA, 18 bp, Artificial Sequence
  Description of Artificial Sequence Synthetic oligonucleotide
tcctagatggaccgctgt

SEQ ID NO: 89 - RNA, 21 nt, Artificial Sequence
  Description of Artificial Sequence Synthetic siRNA sequence
caggugucuaggaauucgcuu

SEQ ID NO: 90 - RNA, 21 nt, Artificial Sequence
  Description of Artificial Sequence Synthetic siRNA sequence
cugugcagucacucaccaguu

SEQ ID NO: 91 - RNA, 21 nt, Artificial Sequence
  Description of Artificial Sequence Synthetic siRNA sequence
cuagguuggggucgcaguauu

(19) United States
(12) Patent Application Publication
(10) Pub. No.: US 2006/0216343 A1
(43) Pub. Date: Sep. 28, 2006

(54) Pharmaceutical compositions comprising an oligonucleotide as an active agent

(76) Inventors: Steffen Panzner, Halle (DE); Una Rauchhaus, Halle (DE); Gerold Endert, Halle (DE); Stefan Fankhaenel, Dresden (DE)

Correspondence Address: MINTZ LEVIN COHN FERRIS GLOVSKY & POPEO, 666 THIRD AVENUE, NEW YORK, NY, 10017, US

(21) Appl. No.: 11/266,999

(22) Filed: Nov. 4, 2005

Related U.S. Application Data
(60) Provisional application No. 60/625,195, filed on Nov. 5, 2004.
(60) Provisional application No. 60/629,600, filed on Nov. 19, 2004.
(60) Provisional application No. 60/717,293, filed on Sep. 15, 2005.

(30) Foreign Application Priority Data
Nov. 5, 2004 (DE) 10 2004 054 731.9
Nov. 19, 2004 (DE) 10 2004 056 659.3
Sep. 15, 2005 (EP) EP 05 020 218.3

(51) Int. Cl. A61K 48/00; A61K 9/127; C12N 15/88

(52) U.S. Cl. 424/450; 435/458; 514/44

(57) ABSTRACT

A pharmaceutical composition is disclosed, which composition comprises an oligonucleotide as an active agent, the oligonucleotide being adapted to target nucleic acids encoding CD40 thereby to modulate the expression of CD40 in mammalian cells, and a liposome as an excipient. Said liposome is an amphoteric liposome. Also disclosed is a method for the treatment or prophylaxis of a disease or condition associated with the expression of CD40 in a human or non-human animal patient by administering to said patient a therapeutically or prophylactically effective amount of such a composition.

This application claims priority to German Patent Application No. DE 10 2004 054 731.9, filed Nov. 5, 2004; German Patent Application No. DE 10 2004 056 659.3, filed Nov. 19, 2004; and European Patent Application No. EP 05 020 218.3, filed Sep. 15, 2005. The application also claims the benefit of U.S. Patent Application Ser. No. 60/625,195, filed Nov. 5, 2004; U.S. Patent Application Ser. No. 60/629,600, filed Nov. 19, 2004; and U.S. Patent Application Ser. No. 60/717,293, filed Sep. 15, 2005, the entire contents of which are hereby incorporated by reference herein.

### FIELD OF THE INVENTION

The present invention relates to pharmaceutical compositions comprising an oligonucleotide as an active agent, and has particular reference to such compositions comprising an oligonucleotide that is adapted to target nucleic acids encoding CD40, thereby to modulate the expression of CD40 in mammalian cells. The invention includes compositions adapted for systemic delivery or for topical application.

### BACKGROUND OF THE INVENTION

CD40 was first described by Pauli, et al. 1984 (Cancer Immunol. Immunotherapy 17: 173-179). The protein is primarily expressed on dendritic cells and B-cells and interacts with its ligand (CD40 ligand or CD154) on T-cells. The signalling between CD40 and CD154 is crucial for the development of a humoral immune response. Overstimulation of the pathway may lead to an immunological imbalance and consequently to a variety of immune-associated disorders, including graft rejection, graft-versus-host disease, multiple sclerosis, systemic lupus erythematosous, rheumatoid arthritis, asthma, inflammatory bowel disease, psoriasis and thyroiditis. CD40 overexpression might also be involved in tumour growth (Gruss, et al. 1997, Leuk Lymphoma. 24(5-6):393-422). CD40 signals into the NF-κB pathway, consequently leading to activation of the transcription factor and the eventual release of cytokines such as IL-1, TNFα and IFNγ, which in turn activate other cells, thus promoting inflammation using a positive feedback mechanism.

Inhibition of the early events in the pathway described above has been proposed as an effective strategy to inhibit immune disorders or inflammation processes. Examples include the competitive binding of TNFα using antibodies, receptor blocking using antibodies against the TNFα-receptor and competitive inhibition of NF-κB binding. Since CD40 signals through its interaction with the trimeric ligand, CD154, inhibition of the signalling event with small molecule inhibitors is unlikely and therapeutic developments have therefore focused on the use of blocking antibodies. More specifically, the CD40/CD154 interaction may be blocked using antibodies targeted against one of the components, as described by Holstager, et al. 2000 (J. Biol. Chem. 275: 15392-15398) or Baccam & Bishop 1999 (Eur. J. Immunol. 29: 3855-3866). However, the CD40 antibodies under development give rise to side reactions, and there is therefore an need for alternative means to cut the inflammatory feedback loop at this point.

Oligonucleotides directed against the mRNA of CD40 offer an alternative approach to interrupt the signalling cascade. Protein expression can be specifically downregulated using oligonucleotides such, for example, as antisense, locked nucleic acids (LNA), peptide nucleic acids (PNA), morpholino nucleic acids (Morpholinos) and small interfering RNAs (siRNA) of various chemistries.

A number of sequences targeted against CD40 mRNA have been validated in vitro so far. US 2004/0186071 and U.S. Pat. No. 6,197,584, both to Bennett, et al., for example, give a detailed description of such oligonucleotides based on antisense mechanisms. Pluvinet, et al. in Blood, 2004 first described the down-regulation of CD40 using siRNA against the human target. Further, WO 2004/090108 to Manoharan describes the applicability of novel oligonucleotides to inhibit the expression of CD40 protein. Indirect means to downregulate the CD40 expression are described in DE 10049549 to Hecker and Wagner, using the inhibition of transcription factor IFR-1.

However, in vivo proof of the concept has not previously been disclosed, and poor delivery of the active oligonucleotides is assumed to be the most likely reason. It is known in the art that oligonucleotides, irrespective of their actual chemical origin, may lack therapeutic efficacy owing to their instability in body fluids or inefficient uptake into cells or both. Chemically modified oligonucleotides such, for example, as the above-mentioned variants or conjugates with ligands or polymers represent one strategy for overcoming practical limitations. A second set of strategies comprehends the use of carrier systems, in particular liposomes, for the protection, targeting and enhanced uptake of oligonucleotides into cells.

For use as such a carrier system, a liposome should desirably show a high encapsulation efficiency and be economical to produce; it should have a good colloidal stability and provide an enhanced uptake of drug into cells; it should also have a low toxicity and immunogenicity. Liposomes for systemic delivery should also be stable in human serum. It is known that serum components, particularly complement, may perforate lipid membranes, thereby causing the release of encapsulated drug. The extent to which such release occurs depends upon the composition of the membrane concerned and the molecular size of the drug encapsulated therein. Thus, small molecules may be released rapidly, whilst large molecules such as plasmids may not be affected at all.

Anionic or neutral liposomes often possess excellent colloidal stability, since no aggregation occurs between the carrier and the environment. Consequently their biodistribution is excellent, and their potential for irritation and cytotoxicity is low. However, such carriers frequently lack encapsulation efficiency and do not provide an endosomolytic signal that facilitates further uptake into cells (Journal of Pharmacology and experimental Therapeutics (2000), 292, 480-488 by Klimuk, et al.).

A great many of publications deal with cationic liposomal systems, e.g. Molecular Membrane Biology (1999), 16, 129-140 by Maurer, et al.; BBA (2000) 1464, 251-261 by Meidan, et al.; Reviews in Biology and Biotechnology (2001), 1(2), 27-33 by Fiset & Gounni. Although cationic systems may provide high loading efficiencies, they lack colloidal stability, in particular after contact with body fluids. Ionic interactions with proteins and/or other biopolymers may lead to aggregate formation with the extracellular matrix or with cell surfaces in situ. Cationic lipids have also often been found to be toxic as shown by Filion, et al. in BBA (1997), 1329(2), 345-356; Dass in J. Pharm. Pharmacol. 2002), 54(5), 593-601; Hirko, et al. in Curr. Med. Chem., 10(14), 1185-1193.

Attempts have been made to overcome such limitations by the addition of components that stabilise the carriers sterically. Polyethyleneglycols of various chain lengths, for example, are known to ameliorate the aggregation problems associated with the use of cationic components in body fluids, and PEGylated cationic liposomes show enhanced circulation times in vivo (BBA (2001) 1510, 152-166 by Semple, et al.). Nevertheless, the use of PEG does not solve the intrinsic toxicity problem associated with cationic lipids. It is also known that PEG may substantially inhibit the productive entry of such liposomes into cells or their intracellular delivery (Song, et al. in BBA (2002), 1558(1), 1-13).

Amphoteric liposomes are a recently described class of liposomes having an anionic or neutral charge at pH 7.5 and a cationic charge at pH 4. Reference is made here to WO 02/066490, WO 02/066012 and WO 03/070735, all to Panzner, et al., which are incorporated herein by reference and give a detailed description of amphoteric liposomes. Further disclosures are made in WO 03/070220 and WO 03 070735, also to Panzner, et al., which are incorporated herein by reference and describe further pH sensitive lipids for use in the manufacture of such amphoteric liposomes.

Amphoteric liposomes have an excellent biodistribution and are well tolerated in animals. They can encapsulate nucleic acid molecules with high efficiency.

In summary, CD40 represents an attractive target for the treatment of inflammatory or immune disorders which potentially can be alleviated using oligonucleotide inhibitors such, for example, as antisense or siRNA molecules. However, it has not been possible hitherto to employ such active oligonucleotides successfully in vivo.

### OBJECTS OF THE INVENTION

An object of the present invention therefore is to provide a pharmaceutical composition comprising an oligonucleotide that is directed against CD40.

A particular object of the present invention is to provide such a composition for topical treatment.

Another particular object of the present invention is to provide such a composition that may be administered systemically. Desirably, such composition should not release its oligonucleotide prematurely upon contact with serum or should at least release its contents only slowly.

A different object of the present invention is to provide a method of treating or preventing an inflammatory, immune or autoimmune disorder of a human or non-human animal.

Yet another object of the present invention is to provide a method for preventing or treating graft rejection, graft-versus-host disease, multiple sclerosis, systemic lupus erythematosous, rheumatoid arthritis, asthma, inflammatory bowel disease, psoriasis or thyroiditis.

Yet another object of the present invention is to provide a method for preventing or treating graft rejection, graft-versus-host disease, inflammatory bowel disease, Morbus Crohn or Colitis ulcerosa.

Yet another object of the present invention is to provide a pharmaceutical composition that is suitable for the topical treatment of inflamed regions of the intestine, the lungs or the skin.

### SUMMARY OF THE INVENTION

According to one aspect of the present invention therefore there is provided a pharmaceutical composition comprising an oligonucleotide as an active agent, which oligonucleotide is adapted to target nucleic acids encoding CD40 thereby to modulate the expression of CD40 in mammalian cells, and a liposome as an excipient; characterised in that said liposome is an amphoteric liposome.

Preferably said oligonucleotide is directed against human CD40.

The pharmaceutical composition of the present invention is generally suitable for local administration and may further comprises a vehicle and be formulated for local administration. Serum-stable embodiments may also be employed for systemic delivery, and in such embodiments, said composition may further comprise a vehicle and be formulated for systemic delivery.

Said amphoteric liposomes may be negatively charged or neutral at pH 7.4 and cationic at pH 4. Preferably, a substantial proportion, or all, of said oligonucleotide is physically entrapped within the amphoteric liposomes. The liposomes may have a size in the range 50 to 500 nm, preferably 100 to 500 nm, more preferably 150 and 300 nm.

In a different aspect of the present invention there is provided a method for the treatment or prophylaxis of a disease or condition associated with the expression of CD40 in a human or non-human animal patient by administering to said patient a therapeutically or prophylactically effective amount of a composition in accordance with the present invention.

By “amphoteric” is meant herein that the liposomes comprise charged groups of both anionic and cationic character wherein:

(i) at least one of the charged groups has a pKa between 4 and 7.4,

(ii) the cationic charge prevails at pH 4 and

(iii) the anionic charge prevails at pH 7.4;

whereby the liposomes have an isoelectric point of zero net charge between pH 4 and pH 7.4. Amphoteric character, by this definition, is different from “zwitterionic character”, because zwitterions do not have a pKa in the range mentioned above. In consequence, zwitterions are essentially neutral over a range of pH values. Phosphatidylcholine or phosphatidylethanolamines, for example, are neutral lipids with zwitterionic character.

Following is a description by way of example only with reference to the accompanying drawings of embodiments of the present invention.

### BRIEF DESCRIPTION OF THE DRAWINGS

FIG. 1: Microscopic scoring of colonic damage.
Control control animals, PBS treated
CD40/0 treated at day 0, 4 h prior induction
CD40/0_{—}3 treated at day 0, 4 k prior induction and day 3
SCR/0 treated with scrambled control, 4 h prior induction
CD40/3 treated at day 3 only
SCR/3 treated with scrambled control at day 3

FIGS. 2A-D: Colon sections after various treatments.
A normal, unaffected bowel wall
B inflamed, but untreated bowel wall
C treatment prior colitis induction using the scrambled control
D treatment prior colitis induction using the specific CD40 antisense

FIG. 3: Perfusion index (filled columns) and stasis index (open columns) in allogeneic control (control), CD40 antisense ODN (AS) or scrambled control ODN (SCR) treated grafts 7 days post transplantation (n=3). * p<0.05 vs. control, # p<0.05 AS vs. SCR

FIG. 4: Functional capillary density (FCD) in the mucosa in allogeneic control (control), CD40 antisense ODN (AS) or scrambled control ODN (SCR) treated grafts 7 days post transplantation (n=3). * p<0.05 vs. control, # p<0.05 AS vs. SCR

FIG. 5: Red blood cell velocity (RBCV) in submucosal vessels of small bowel transplants in allogeneic control (control), CD40 antisense ODN (AS) or scrambled control ODN (SCR) treated grafts 7 days post transplantation (n=3). * p<0.05 vs. control, # p<0.05 AS vs. SCR

FIG. 6: Leukocyte-endothelial cell interaction in allogeneic control (control), CD40 antisense ODN (AS) or scrambled control ODN (SCR) treated grafts 7 days post transplantation (n=3). * p<0.05 vs. control, # p<0.05 AS vs. SCR

FIG. 7: Joint swelling of rats after treatment with free or liposomal CD40-ODN. Swelling is expressed as the difference in size between the right and left knee joint. Animals were treated as described and three injections were given at 6, 48 and 96 hrs.

FIG. 8: Body weights [g] of rats 21 days after the application of free CD40-ODN or liposomal CD40-ODN. Animals were treated as described and three injections were at 6, 48 and 96 hrs.

FIG. 9: Organ weights [g] of spleen and thyme of rats 21 days after the application of free CD40-ODN or liposomal CD40-ODN

FIG. 10: Liver weights [g] of rats 21 days after the application of free CD40-ODN or liposomal CD40-ODN

FIG. 11: Porcine CD40 cDNA sequence (SEQ ID NO:4) for targeting in accordance with the present invention.

### DETAILED DESCRIPTION OF THE INVENTION

In some embodiments of the present invention, said amphoteric liposomes may be formed from a lipid phase comprising an amphoteric lipid. Said lipid phase may comprise 5 to 30 mol. % of said amphoteric lipid, preferably 10 to 25 mol. %.

Suitable amphoteric lipids are disclosed in WO 02/066489 and WO 03/070735. Preferably, said amphoteric lipid is selected from the group consisting of HistChol, HistDG, isoHistSuccDG, Acylcarnosin and HCChol. (A glossary of such abbreviated forms of the names of the lipids referred to herein is included below for ease of reference. A number of such abbreviations are those that are commonly used by those skilled in the art.)

A particularly preferred amphoteric lipid is HistChol.

Alternatively, said amphoteric liposomes may be formed from a lipid phase comprising a mixture of lipid components with amphoteric properties. Such amphoteric liposomes may be formed from pH-responsive anionic and/or cationic components, as disclosed for example in WO 02/066012. Cationic lipids sensitive to pH are disclosed in WO 02/066489 and WO 03/070220 and in the references made therein, in particular in Budker, et al. 1996, Nat Biotechnol. 14(6):760-4, and can be used in combination with constitutively charged anionic lipids or with anionic lipids that are sensitive to pH.

Alternatively, the cationic charge may be introduced from constitutively charged lipids that are known to those skilled in the art in combination with a pH sensitive anionic lipid.

Combinations of constitutively charged anionic and cationic lipids, e.g. DOTAP and DPPG, are not preferred. Thus, in some presently preferred embodiments of the invention, said mixture of lipid components may comprise (i) a stable cationic lipid and a chargeable anionic lipid, (ii) a chargeable cationic lipid and chargeable anionic lipid or (iii) a stable anionic lipid and a chargeable cationic lipid.

Preferred cationic components include DPIM, CHIM, DORIE, DDAB, DAC-Chol, TC-Chol, DOTMA, DOGS, (C18)₂Gly⁺N,N-dioctadecylamido-glycin, CTAB, CPyC, DODAP and DOEPC.

Further preferred cationic lipids are DMTAP, DPTAP, DOTAP, DC-Chol, MoChol and HisChol.

Preferred anionic lipids for use with the invention include DOGSucc, POGSucc, DMGSucc, DPGSucc, DMPS, DPPS, DOPS, POPS, DMPG, DPPG, DOPG, POPG, DMPA, DPPA, DOPA, POPA, CHEMS and CetylP.

Particularly preferred anionic lipids are DOGSucc, DMGSucc, DMPG, DPPG, DOPG, POPG, DMPA, DPPA, DOPA, POPA, CHEMS and CetylP.

Preferably, such an amphoteric mixture of lipids does not constitute more than about 70 mol. % of the lipid phase. In some embodiments, said mixture may constitute not more than 50 mol. % of the lipid phase; preferably said lipid phase comprises about 20 to about 40 mol. % of such a mixture.

In some embodiments, said lipid phase may further comprise a neutral lipid, preferably a neutral phospholipid, such as a phosphatidylcholine. Presently preferred phosphatidylcholines include POPC, natural or hydrogenated soy bean PC, natural or hydrogenated egg PC, DMPC, DPPC, DSPC and DOPC.

More preferably, said phosphatidylcholine comprises POPC, non-hydrogenated soy bean PC or non-hydrogenated egg PC.

The lipid phase may comprise at least 15 mol. % of said phosphatidylcholine, preferably at least 20 mol. %. In some embodiments, said lipid phase may comprise no less than about 25 mol. % phosphatidylcholine. Alternatively, said lipid phase may comprise no less than about 40 mol. % phosphatidylcholine.

A presently preferred composition in accordance with the present invention comprises a liposome having the formulation 60 mol. % POPC, about 10 mol. % DOTAP and about 30 mol. % CHEMS.

Said neutral lipid may comprise a phosphatidylethanolamine or a mixture of phosphatidylcholine and phosphatidylethanolamine. Said neutral phosphatidylcholines or phosphatidylethanolamines or mixtures of the two may be present in the lipid phase in the molar amount (mol. %) not constituted by the other components of the lipid phase, but to at least 20 mol. % (the total for the lipid phase being 100 mol. %).

Preferred phosphatidylethanolamines include DOPE, DMPE and DPPE.

In some embodiments said neutral lipid may comprise POPC and DOPE.

Advantageously, said lipid phase may comprise a mixture of anionic and cationic lipids with amphoteric properties, phosphatidylcholine and phosphatidylethanolamine. It has been found that amphoteric liposomes formed from such a lipid phase may be serum-stable and therefore suitable for systemic delivery. Preferably said lipid phase comprises MoChol as a cationic lipid and CHEMS or DMG-Succ as an anionic lipid.

Further presently preferred amphoteric liposomes for use as the excipient in the composition of the present invention have the following formulations:
(a) about 15 mol. % POPC, about 45 mol. % DOPE, about 20 mol. % MoChol and about 20 mol. % CHEMS;
(b) about 10 mol. % POPC, about 30 mol. % DOPE, about 30 mol. % MoChol and about 30 mol. % CHEMS;
(c) about 10 mol. % POPC, about 30 mol. % DOPE, about 20 mol. % MoChol and about 40 mol. % CHEMS;
(d) about 6 mol. % POPC, about 24 mol. % DOPE, about 47 mol. % MoChol and about 23 mol. % CHEMS.

In some embodiments, said liposome may further comprise neutral phosphatidylcholines and cholesterol. Such liposomes may also be serum-stable.

Alternatively, a serum-stable liposome suitable for systemic delivery may comprise an amphoteric lipid or a mix of lipid components with amphoteric properties, cholesterol and a neutral lipid, such as phosphatidylcholine. In some embodiments, said lipid phase may comprise from 30 mol. % to 50 mol. % cholesterol, preferably from about 35 mol. % to about 45 mol. %. Alternatively, said lipid phase may comprise phosphatidylcholine and from 10 mol. % to 25 mol. % cholesterol, preferably from about 15 mol. % to about 25 mol. %.

A presently preferred formulation comprises 10 to 25 mol. % amphoteric lipid, e.g. HistChol, HistDG or Acylcarnosin, 15 to 25 mol. % cholesterol and the remainder being POPC, soy bean PC, egg PC, DMPC, DPPC or DOPC, preferably POPC; for example about 60 mol. % POPC, about 20 mol. % HistChol and about 20 mol. % Chol

Another presently preferred composition in accordance with the present invention comprises a liposome including a mix of lipid components with amphoteric properties and having the formulation about 30 mol. % POPC, about 10 mol. % DOTAP, about 20 mol. % CHEMS and about 40 mol. % Chol.

The pharmaceutical composition of the present invention comprises an oligonucleotide that targets nucleic acids encoding CD40, thereby to attenuate the expression of such CD40 in mammalian cells. By “nucleic acids encoding CD40” is meant herein DNA coding for CD40, as well as RNAs derived from such DNA, being pre-mRNA or mRNA. Specific hybridisation between the target nucleic acid and one or more oligonucleotides directed against such a sequence as the active agent may result in inhibition of CD40 expression. To achieve such specific targeting, said oligonucleotide should preferably comprise a continuous stretch of nucleotides that is complementary to the sequence of the target nucleic acid. The oligonucleotide may vary in length between as little as 10, preferably 15, and even more preferably 18, and 50, preferably 30, and more preferably 25 nucleotides. The fit between the oligonucleotide and the target sequence is preferably perfect with each base of the oligonucleotide forming a base pair with its complementary base on the target nucleic acid over a continuous stretch of the abovementioned number of oligonucleotides. The pair of sequences may in some embodiments contain one or a few mismatches within said continuous stretch of base pairs, although this is less preferred.

Oligonucleotides fulfilling the abovementioned criteria may have a range of different chemistries and/or topologies. Oligonucleotides may be single stranded or double stranded. Single stranded oligonucleotides include, but are not limited to, DNA-based oligonucleotides, locked nucleic acids and 2′-modified oligonucleotides, commonly known as antisense oligonucleotides. Backbone or base modifications may include, but are not limited to, phosphothioate DNA (PTO), 2′O-methyl RNA (2′Ome), 2′O-methoxyethyl-RNA (2′MOE), peptide nucleic acids (PNA), N3′-P5′ phosphoamidates (NP), 2′fluoroarabino nucleic acids (FANA), locked nucleic acids (LNA), morpholine phosphoamidate (Morpholino), cyclohexene nucleic acid (CeNA) and tricyclo-DNA (tcDNA). Moreover, mixed chemistries are known in the art, being constructed from more than a single nucleotide species such, for example, as copolymers, block-copolymers and gapmers.

In addition to the aforementioned oligonucleotides, CD40 expression may also be inhibited using double stranded RNA molecules containing complementary sequence motifs. Such RNA molecules are known in the art as siRNA molecules. Again, various chemistries are adapted to this class of oligonucleotides. Further, DNA/RNA hybrid systems are known in the art.

More specifically, reference is made here to U.S. Pat. No. 6,197,584 and US 2004/0186071, both to Bennett, which describe useful sequences and chemistries of such oligonucleotides. Reference is also made to Pluvinet, et al. in Blood, 2004, describing siRNA sequence motifs for the inhibition of CD40. Further siRNA motifs are in public domain and can be obtained, e.g. from Santa Cruz Biotechnology (Santa Cruz, U.S.A.).

The pharmaceutical composition of the present invention may be formulated for use as a colloid in a suitable pharmacologically acceptable vehicle. Vehicles such as water, saline, phosphate buffered saline and the like are well known to those skilled in the art for this purpose.

In some embodiments, the composition of the present invention may be administered at a physiological pH of between about 7 and about 8. To this end, the composition comprising the active agent, excipient and vehicle may be formulated to have a pH in this range.

The composition of the invention may be manufactured using suitable methods that are known to those skilled in the art. Such methods include, but are not limited to, extrusion through membranes of defined pore size, injection of lipid solutions in ethanol into a water phase containing the cargo to be encapsulated, or high pressure homogenisation.

A solution of the oligonucleotide may be contacted with said excipient at a neutral pH, thereby resulting in volume inclusion of a certain percentage of the solution. An high concentrations of the excipient, ranging from about 50 mM to about 150 mM, is preferred to achieve substantial encapsulation of the active agent.

Amphoteric liposomes used as the excipient in accordance with the present invention offer the distinct advantage of binding oligonucleotides at or below their isoelectric point, thereby concentrating said active agent at the liposome surface. This process is described in more detail in WO 02/066012.

Irrespective of the actual production process used to make the composition of the invention, in some embodiments, non-encapsulated oligonucleotide may be removed from the liposomes after the initial production step in which the liposomes are formed as tight containers. Again, the technical literature and the references included herein describe such methodology in detail and suitable process steps may include, but are not limited to, size exclusion chromatography, sedimentation, dialysis, ultrafiltration and diafiltration.

However, the removal of any non-encapsulated oligonucleotide is not required for performance of the invention, and in some embodiments the composition may comprise free as well as entrapped drug.

Following are particular combinations of process steps that may be used advantageously for preparing pharmaceutical compositions in different embodiments of the present invention:

(A)

I. encapsulation of the active agent at neutral pH using a concentration of said active agent of between about 0,5 mg/mL and about 50 mg/mL, preferably between about 1 and about 20 mg/mL, and an excipient concentration of between about 50 mM and about 150 mM.
II. said vehicle may be water, saline or buffered saline
III. actual liposome formation and sizing step
IV. non-entrapped active agent is not removed
V. optional lyophilisation and reconstitution with water
VI. storage form: suspension or lyophilised powder
VII. administration at neutral pH
   (B)
I. encapsulation of the active agent at neutral pH using a concentration of active agent of between about 0,5 mg/mL and about 50 mg/mL, preferably between about 1 and about 20 mg/mL, and an excipient concentration of between about 50 mM and about 150 mM.
II. the vehicle may be water, saline or buffered saline
III. actual liposome formation and sizing step
IV. non-entrapped drug removed
V. storage form: suspension
VI. administration at neutral pH

The present invention therefore comprehends a pharmaceutical composition comprising an oligonucleotides directed against CD40 as an active agent and an amphoteric liposome as an excipient. Such formulations have been found to be therapeutically active in the treatment of inflammations and autoimmune disorders, and accordingly the invention further comprehends the use of the composition of the invention for the prevention or treatment of inflammations, immune or autoimmune disorders, including graft rejection, graft-versus-host disease, multiple sclerosis, systemic lupus erythematosous, rheumatoid arthritis, asthma, asthma bronchiale, inflammatory bowel disease, psoriasis, thyroiditis, Morbus Crohn, Colitis ulcerosa, COPD and atopic dermatitis.

As mentioned above, in some embodiments, the composition of the present invention may be substantially stable in serum and accordingly, in such embodiments, the composition may be delivered systemically in mammals, especially humans.

The pharmaceutical composition of the present invention may also be used for topical treatments, for example the treatment of inflamed mucosa. In particular, the composition of the invention may be used for the treatment or prophylaxis of inflammatory bowel disease or graft rejection. The composition of the present invention may also be adapted for topical application to the skin or lungs.

Administration of the pharmaceutical composition is within the skill of those skilled in the art. Dosing may be dependent upon the severity and/or responsiveness of the disease to be treated, the course of treatment lasting from several days to several months, or until cure has been effected or diminution of the symptoms of the disease has been achieved. Optimal dosing schedules may be calculated from measurements of drug accumulation in the body of the patient. Those of ordinary skill in the art can easily determine optimum dosages, dosing methodologies and repetition rates. Optimum dosages may vary depending on the relative potency of the individual oligonucleotides in the composition of the invention and can generally be estimated based on EC50 values found to be effective in animal models. In general, a unit dosage may be from about 0.01 μg to about 20 mg oligonucleotide of kg body weight and may be given daily, weekly, monthly or yearly or even less regularly. Persons of ordinary skill in the art can easily estimate repetition rates for dosing based on measured residence times and concentrations of the drug in body fluids or tissues. Following successful treatment, it may be desirable to have the patient undergo maintenance therapy to prevent the recurrence of the disease state, wherein the formulation may be administered at maintenance doses, ranging from about 0.01 μg to about 20 mg oligonucleotide per kg of body weight, once or more daily to once in a year.

### EXAMPLES

### Example 1

### Preparation of CD40-ODN-Containing Liposomes

A mixture of 85 μmol POPC, 42 μmol CHEMS and 14 μmol DOTAP was dissolved in chloroform and evaporated in a round bottom flask to dryness under vacuum.

ODN with the sequence T*C*C*TAGATGGACCGCT*G*T was used with asterisks indicating a phosphorothioate linkage between the nucleotides (after Gao, Ph.D. thesis, Goettingen 2003, rAS3).

Lipid films were hydrated with 1 mg ODN in 1 mL of buffer (10 mM sodium acetate, 150 mM NaCl pH 4.5). The suspensions were hydrated for 25 minutes in a water bath at room temperature, sonicated for 5 minutes and eventually frozen at −70° C. After thawing the liposomal suspensions were extruded 15 times through polycarbonate membranes with a pore size of 400 nm. The liposome suspensions were brought to pH 7.5 using 1M HEPES buffer and to 0.8 M sucrose using a stock solution. Non-encapsulated ODN was removed from the extruded sample by flotation through 0.5 M sucrose overlaid with 10 mM HEPES, 150 mM NaCl pH 7.5 and the liposome suspension was stored at 4° C. Resulting liposomes were characterised by dynamic light scattering and found to be 220 to 250 nm in size.

### Example 2

### Colitis Induction

Colitis was induced by using a single intra-colonic application of 2,4,6-trinitrobenzene sulphonic acid (TNBS) prepared by adding 20 mg of TNBS to 135 μl of 35% ethanol in 150 mM NaCl. Male Wistar rats (200 . . . 250 g) were placed under light ether anaesthesia and the mixture was administered using an 8 cm long catheter inserted through the anal canal into the descending colon. After removing the catheter, rats were held in a headfirst position for 30s to avoid flowing out of the enema and rats were kept under normal condition afterwards.

### Example 3

### Treatment and Analysis

Rats were treated with CD40 antisense from Example 1 either 4 hours before or 3 days after the colitis induction. The antisense suspension from Example 1 was brought to pH 4.5 using 1M buffered acetic acid/sodium acetate pH 4.0 and a total of 100 μl containing 2,7 μg CD40 antisense suspension was applied to the colon according to Example 2.

Seven days after induction of the colitis the animals were sacrificed. The colon was removed and opened longitudinally. Colon samples were fixed in PBS containing 4% formaldehyde. Paraffin-embedded sections (5 μm) were stained with haematoxylin/eosin followed by microscopic inspection.

Colonic damage was scored according to the following criteria:

TABLE 1
Criteria for microscopic scoring of colonic damage.

| Parameters | Score |
|---|---|
| Ulceration | |
| No | 0 |
| Minor | 1 |
| Major | 2 |
| Inflammation | |
| None | 0 |
| Minor | 1 |
| Major | 2 |
| Severe | 3 |
| Depth of lesion | |
| None | 0 |
| Superficial | 1 |
| One third | 2 |
| Two third | 3 |
| Transmural | 4 |
| Fibrosis | |
| None | 0 |
| Minor | 1 |
| Major | 2 |
| Lymphocyte infiltration | |
| No | 0 |
| Yes | 1 |
| Total score | 0-12 |

The results presented in FIGS. 1 and 2 demonstrate a substantial reduction of the experimental colitis when treated with antisense directed against CD40, but not with the scrambled control antisense. Quite surprisingly, even a single treatment of a fully developed colitis at day 3 resulted in a strong and almost complete reduction of the inflammation. In confirmation to that, prevention of the colitis was also achieved when the formulation was applied in a preventive mode before the initiation of the disease.

### Example 4

### Alternative Formulation

When used as an excipient, a mixture of 60 mol % POPC, 20 mol % HistChol and 20 mol % Cholesterol also resulted in successful treatment of the experimental colitis.

### Example 5

### Non Removal of Outside Antisense

When used as a formulation, non-removal of non encapsulated antisense also resulted in carrier systems that are stable colloids.

### Example 6

### Small Bowel Transplant Treatment

Heterotopic small bowel transplantation was performed in male rats in the allogeneic Brown Norway (RT1n) to Lewis (RT11) strain combination without immunosuppressant therapy.

After explantation of the small bowel and flushing of the graft vessels with Ringer solution, one group of animals (n=3) received donor small bowel transplants pre-treated with the CD40 antisense ODN (group A) or the corresponding scrambled control ODN (group B) formulated in excipient as described in Example 1. The antisense suspension from example 1 was brought to pH 4.5 using 1M buffered acetic acid/sodium acetate pH 4.0.

Donor blood vessels were pre-treated with 2 ml Ringer solution containing CD40 antisense or scrambled control ODN (2.7 μg DNA in a total volume of 100 μl). The bowel lumen was rinsed with UW (University of Wisconsin) solution. After 2 hours of cold ischemia, the DNA solution was flushed out and the grafts were implanted and analyzed histologically.

Analysis

To characterise overall mucosal perfusion, a perfusion index (PI) was calculated by using the equation
PI(%)=(Vp+0.5×Vip)/Vt
where Vp represents the number of perfused villi, Vip the number of all irregularly perfused villi and Vt the total number of villi observed.

To characterise overall mucosal perfusion damage, a stasis index (SI) was calculated as follows:
SI(%)=Vnp/Vt
where Vnp represents the number of non-perfused villi and Vt the total number of all villi observed.

Further analysis of microcirculatory parameters in the mucosa and muscle layers included the assessment of functional capillary density (FCD, length of perfused capillaries per villus area (1/cm) at a magnification of 476×) and red blood cell velocity (RBCV in mm/sec at a magnification of 933×). Analysis of functional capillary density and red blood cell velocity were performed by using the CAPIMAGE software (Zeintl, Heidelberg, Germany), red blood cell velocity was determined by line-to-shift analysis. Furthermore, by using the fluorescent marker Rhodamine 6G adherent leukocytes were identified in each vessel segment (100 μm) and counted as cells that did not move or detach from the endothelium within an observation period of 30 s. Their number was calculated from the diameter and length of the blood vessel, assuming a cylindrical geometry, and expressed as number of cells per mm².

Results

Microcapillary Perfusion of villi within the Graft Mucosa

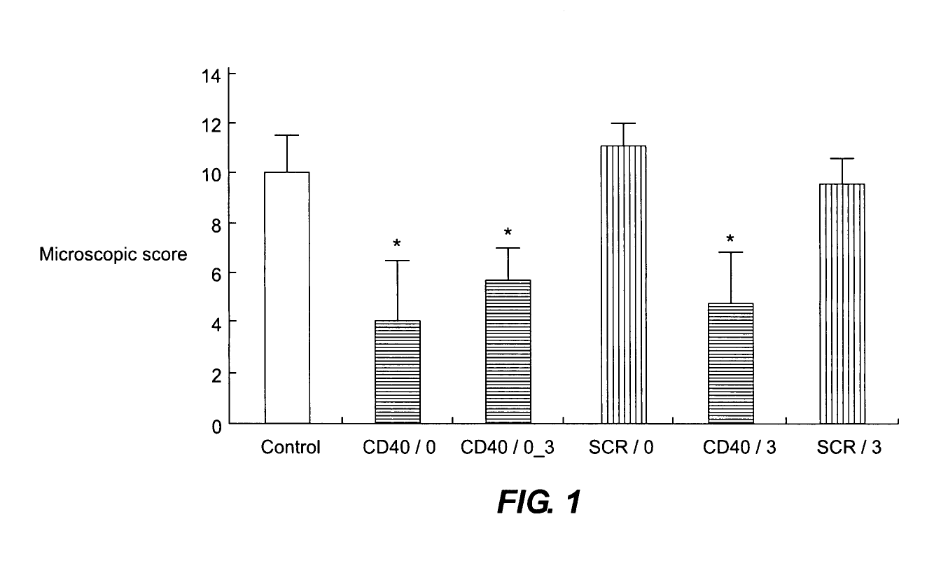

Overall villi perfusion in the graft mucosa was significantly improved in CD40 antisense ODN-treated transplants as compared to the untreated control or scrambled control ODN, respectively. This was shown by the perfusion index representing the percentage of perfused villi in respect to the observed villi per observation field (FIG. 3). Conversely the stasis index, a marker of the percentage of non-perfused villi in respect to the total number of villi per observation field, was significantly reduced in CD40 antisense ODN treated grafts compared to the untreated control and scrambled control ODN, respectively (FIG. 3).

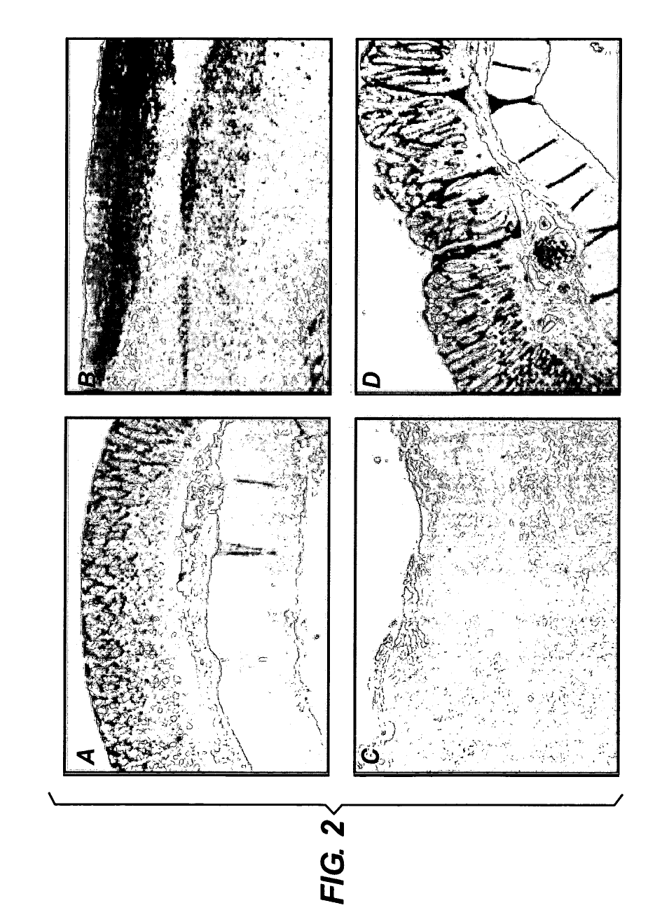

A more detailed analysis of single villus perfusion by measuring the functional capillary density within a single villus per villus area showed again a significantly higher density of perfused capillaries in CD40 antisense ODN treated animals compared to the untreated control and scrambled control ODN, respectively (FIG. 4). This demonstrates a better preserved villus perfusion and hence better mucosal function CD40 antisense ODN treated animals.

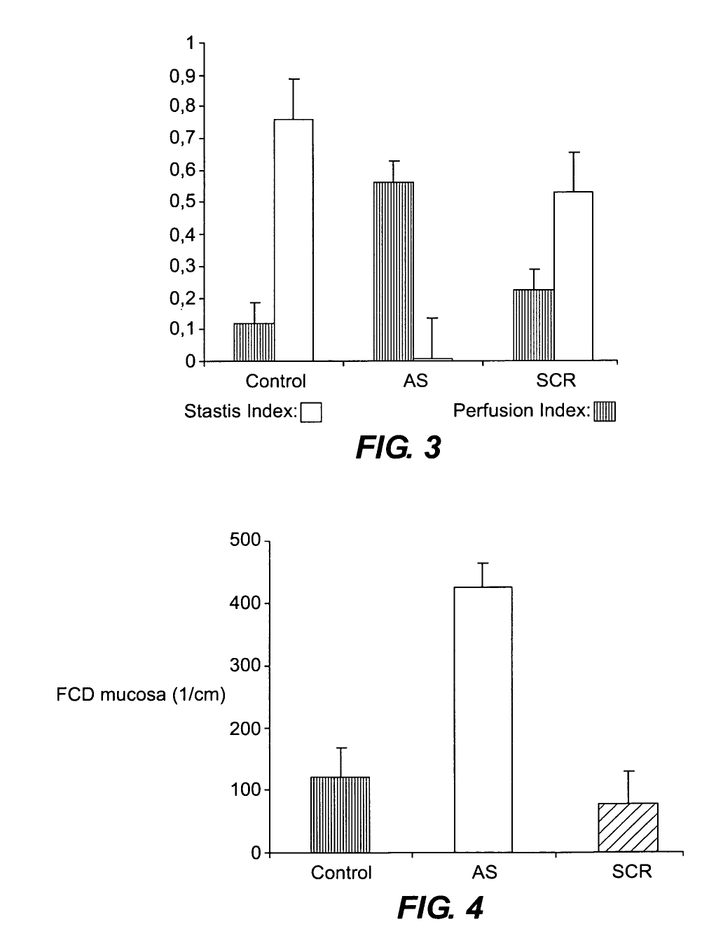

Accordingly, measuring red blood cell velocity in the villus capillaries revealed a significantly greater velocity in CD40 antisense ODN treated transplants compared to the untreated control and scrambled control ODN, respectively (FIG. 5).

Leukocyte-Endothelial Cell Interaction

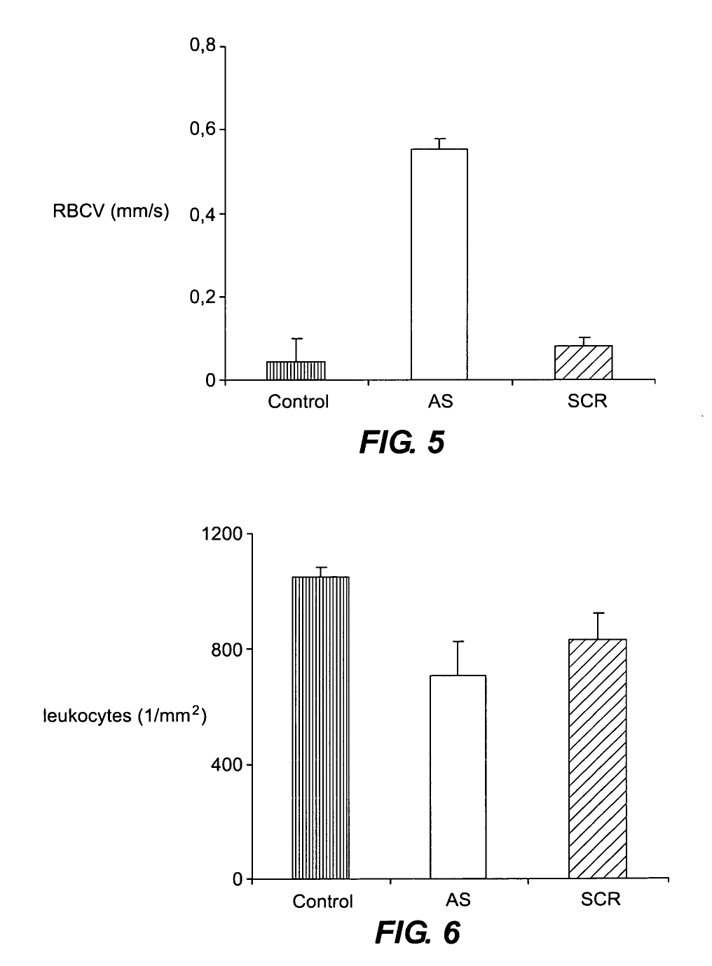

In contrast, evaluation of leukocyte-endothelial cell interaction in submucosal postcapillary venules revealed no significant differences in the number of sticking leukocytes to the endothelial surface between the different treatment and control groups (FIG. 6).

### Example 7

### Preparation of CD40-ODN-Containing Liposomes

A mixture of 30 mol % POPC, 10 mol % DOTAP, 20 mol % Chems and 40 mol % Chol was dissolved in chloroform and evaporated in a round bottom flask to dryness under vacuum.

ODN with the sequence TCCTAGATGGACCGCTGT was purchased from Biognostik GmbH, Germany with full phosphorothioate nucleotide chemistry (after Gao, Ph.D. thesis, Goettingen 2003, rAS3).

The lipid film was hydrated with such amount of ODN solution (20 mg per ml CD40 ODN (18 mer, fully phosphothioated) in 10 mM Hepes, 125 mM NaCl pH 7.5) that the final lipid concentration is 100 mM in the suspension. The suspension was hydrated for 45 minutes in a water bath at 50° C. and sonicated for 15 minutes. Then, the suspension was frozen 3 times at −70° C. for 30 minutes and thawed at 50° C. for 15 minutes.

The liposomal suspension was extruded 19 times through polycarbonate membranes with a pore size of 400 nm. Non-encapsulated ODN was removed from the extruded sample after dilution with water by sedimentation at for 15 hours at 35000 rpm at 15° C.

Other formulations with encapsulated CD40 ODN were prepared using the same conditions.

TABLE 1
examples for Smarticles formulations which encapsulate CD40 ODN

| | | | | Polydisp. |
|---|---|---|---|---|
| Formulation | Lipid | mol % | size | Index |
| 1 | POPC/DOTAP/Chems/ | 30:10:20:40 | 257.9 | 0.206 |
| | Chol | | | |
| 2 | POPC/DOPE/MoChol/ | 6:24:23:47 | 204.7 | 0.197 |
| | DMG-Succ | | | |

The amount of encapsulated ODN was measured by checking the optical density (OD) by 260 nm. The following amounts of ODN were encapsulated in the different Smarticles formulations.

TABLE 2
encapsulated amount of ODN in different Smarticles formulations

| | | | μg ODN/ | |
|---|---|---|---|---|
| Formu- | | | μmol | Encapsulation |
| lation | Lipid | mol % | lipid | efficacy |
| 1 | POPC/DOTAP/ | 30:10:20:40 | 23.2 | 11.6% |
| | Chems/Chol | | | |
| 2 | POPC/DOPE/ | 6:24:23:47 | 32.41 | 12.80% |
| | MoChol/DMG-Succ | | | |

### Example 8

### Therapeutic Efficacy in Arthritis

Female Lewis rats were immunized 21 and 14 days before induction of arthritis by subcutaneous injections of methylated bovine serum albumin (mBSA) in complete Freund's adjuvant. On day 0, arthritis was induced by intraarticular injection of the antigen (mBSA) in physiological buffer into the right knee joint, whereas the left knee joint was used as non-injected normal control joint.

For the treatment studies either free (unencapsulated) CD40-ODN or liposomal CD40-ODN (formulation 1 of Example 7 above: POPC/DOTAP/Chems/Chol 30:10:20:40) was injected intravenously into the tail vein of rats with established AIA 6, 48 and 96 hours post induction of arthritis. Each dosage contains 3 mg CD40-ODN per kg bodyweight (encapsulated CD40-ODN) or 3 and 15 mg CD40-ODN per kg bodyweight (free CD40-ODN9 and free CD40-ODN45, respectively).

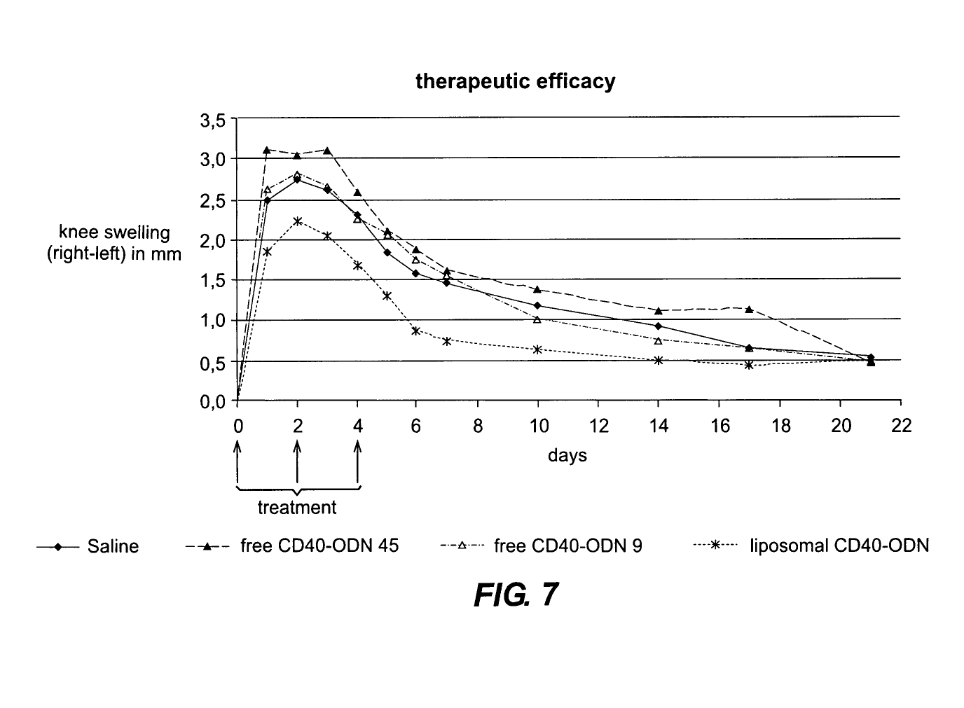

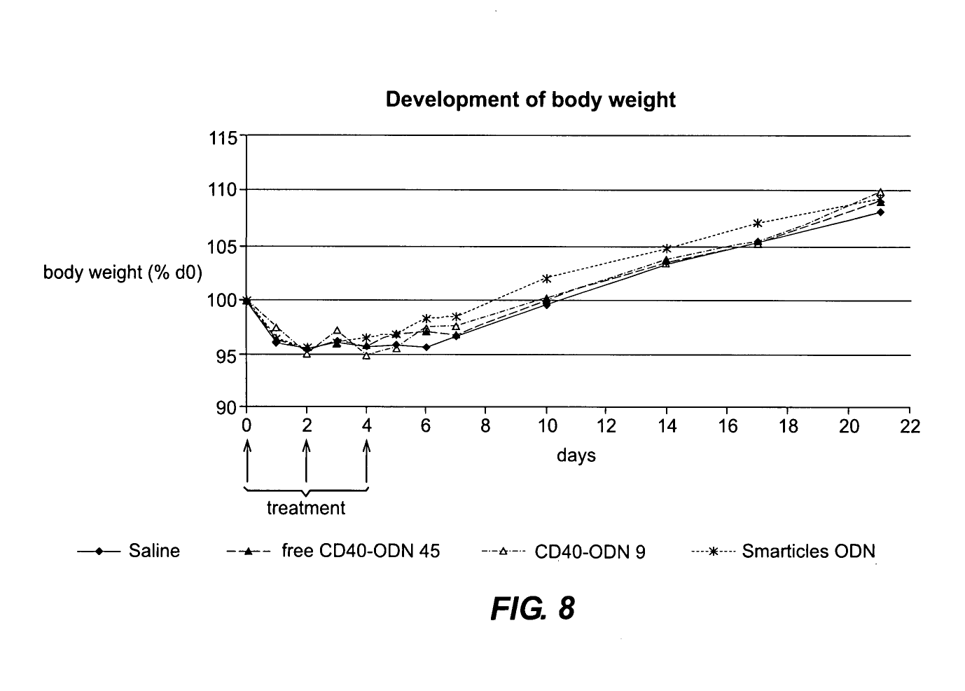

During the experiment the swelling of joints and the body weights of the animals were observed. There was a significant reduction (p<0,05) of the swelling of knee joints over the 21 days after a treatment with encapsulated CD40-ODN (liposomal-ODN, FIG. 7). In contrast treatment with high dose free CD40-ODN (CD40-ODN 45, FIG. 7) resulted in more inflamed knee joint in the acute and the chronic phase of arthritis compared to the saline control. The body weights of all animal groups do not show any discrepancies compared to the saline control (FIG. 8).

### Example 9

### Tolerability

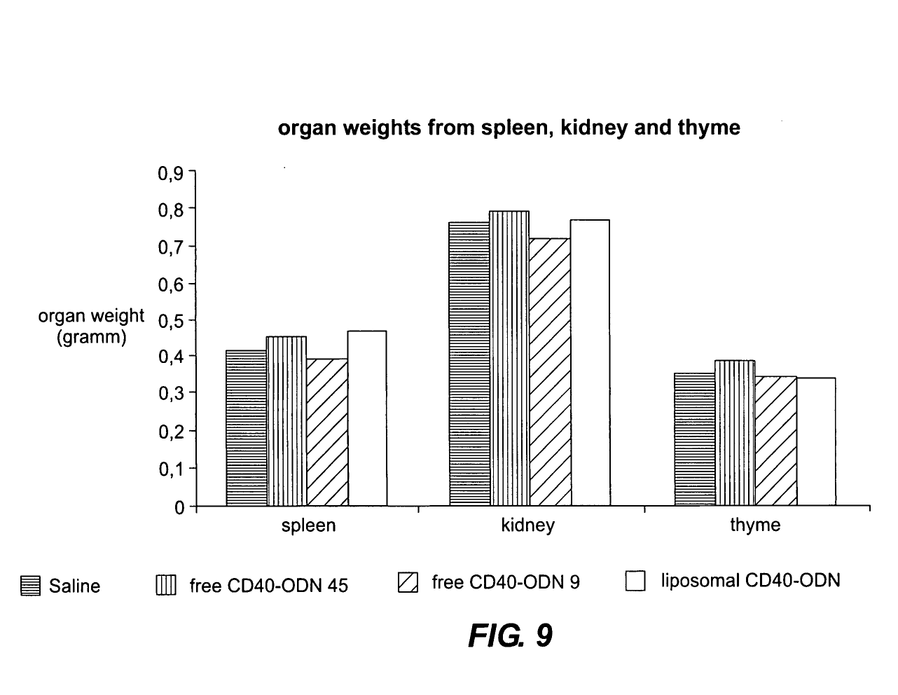

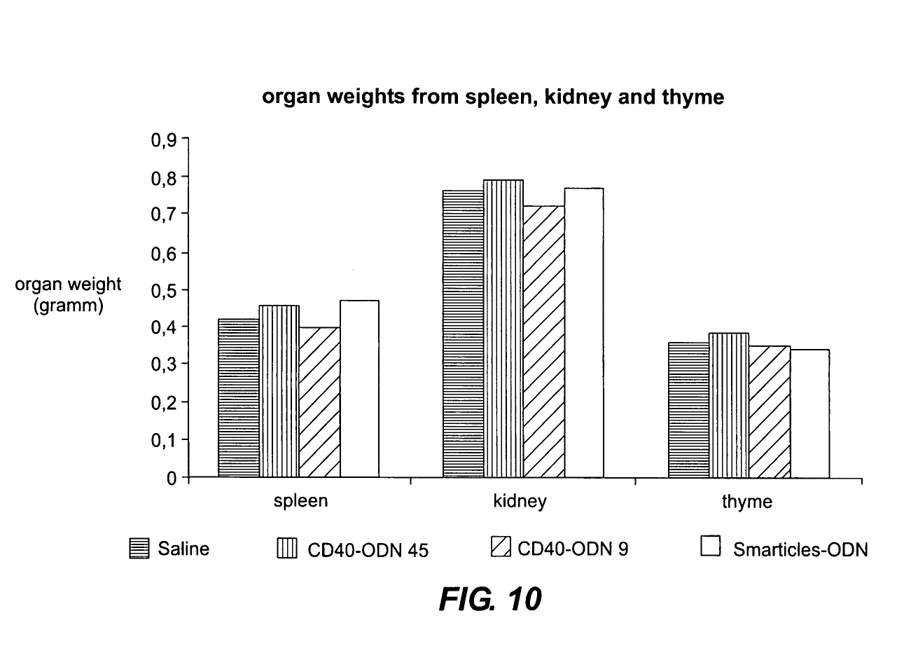

Animals were treated as described in Example 2 and sacrificed on day 21 after the onset of the inflammation. A macroscopic inspection did not reveal any sign of intolerance for the formulations and were found indistinguishable from the control group. In addition, the individual organ weights were measured for liver, spleen, thymus and kidney. A slight reduction in liver weight was observed for the group treated with the liposomal CD40-ODN, all other organ weights were not affected (FIG. 9 and FIG. 10).

### Example 10

### Materials

This example provides non-limiting examples of CD40 nucleotide sequences that may be targeted by oligonucleotides that modulate the expression of CD40 and that are suitable for use in the compositions in accordance with the present invention.

Human CD40 mRNA (GenBank accession no. X60592)

Human CD40 mRNA sequence for targeting in accordance with the present invention is presented in SEQ ID NO:1. Related sequence information is found in published patent application number US 2004/0186071 (i.e., SEQ ID NO:85) to Bennett, et al. and in U.S. Pat. No. 6,197,584 (i.e., SEQ ID NO:85) to Bennett, et al. and in Pluvinet, et al., Blood, 2004, 104(12), 3642-3646, the contents of which are incorporated by reference herein.

| (SEQ ID NO:1): | |
|---|---|
| 1 | gcctcgctcg ggcgcccagt ggtcctgccg cctggtctca cctcgccatg gttcgtctgc |
| 61 | ctctgcagtg cgtcctctgg ggctgcttgc tgaccgctgt ccatccagaa ccacccactg |
| 121 | catgcagaga aaaacagtac ctaataaaca gtcagtgctg ttctttgtgc cagccaggac |
| 181 | agaaactggt gagtgactgc acagagttca ctgaaacgga atgccttcct tgcggtgaaa |
| 241 | gcgaattcct agacacctgg aacagagaga cacactgcca ccagcacaaa tactgcgacc |
| 301 | ccaacctagg gcttcgggtc cagcagaagg gcacctcaga aacagacacc atctgcacct |
| 361 | gtgaagaagg ctggcactgt acgagtgagg cctgtgagag ctgtgtcctg caccgctcat |
| 421 | gctcgcccgg ctttggggtc aagcagattg ctacaggggt ttctgatacc atctgcgagc |
| 481 | cctgcccagt cggcttcttc tccaatgtgt catctgcttt cgaaaaatgt cacccttgga |
| 541 | caagctgtga gaccaaagac ctggttgtgc aacaggcagg cacaaacaag actgatgttg |
| 601 | tctgtggtcc ccaggatcgg ctgagagccc tggtggtgat ccccatcatc ttcgggatcc |
| 661 | tgtttgccat cctcttggtg ctggtcttta tcaaaaaggt ggccaagaag ccaaccaata |
| 721 | aggcccccca ccccaagcag gaaccccagg agatcaattt tcccgacgat cttcctggct |
| 781 | ccaacactgc tgctccagtg caggagactt tacatggatg ccaaccggtc acccaggagg |
| 841 | atggcaaaga gagtcgcatc tcagtgcagg agagacagtg aggctgcacc cacccaggag |
| 901 | tgtggccacg tgggcaaaca ggcagttggc cagagagcct ggtgctgctg ctgcaggggt |
| 961 | gcaggcagaa gcggggagct atgcccagtc agtgccagcc cctc |

Mus Musculus CD40 mRNA

Murine CD40 mRNA sequence for targeting in accordance with the present invention is presented in SEQ ID NO:2. Related sequence information is found in published patent application number US 2004/0186071 (i.e. SEQ ID NO:132) to Bennett, et al., the contents of which are incorporated by reference herein.

| (SEQ ID NO:2): | |
|---|---|
| gcctcctggc ccttcagctg tggtctttcc cgttttctga ctttgcggtg acactgggga | 60 |
| cttccttaga cctctctgga gacgctttcg gttctgcaga gattcccagg ggtattgtgg | 120 |
| gtggggtggg gtaacaatag tgtccctgtg gcgctcccag tccctatagt aatccttcac | 180 |
| ccctctgcta tcttgcaatc aggagagtcc ttagccctgc tataggtggc ttttgaggtc | 240 |
| ctggatgcga ggagggggac tggggggtgg gtcgggtaat gtaagaaaag ggctcctttt | 300 |
| gggaccctgg ctcctccagc caccttggtg cccatccctt aaactcttgg ggacaatcag | 360 |
| actcctggga aggtcctggg gaaatccctg ctcagtgact agccataggc ccaccgcgat | 420 |
| tggtgcccga agaccccgcc ctcttcctgg gcgggactcc tagcagggac tttggagtga | 480 |
| cttgtggctt cagcaggagc cctgtgattt ggctcttctg atctcgccct gcgatggtgt | 540 |
| ctttgcctcg gctgtgcgcg ctatggggct gcttgttgac agcggtgagt ggcttgtgtt | 600 |
| ctaacctcca agggagttag ggcttagaga gtgagagatg gaaagaggaa agaggagaca | 660 |
| agactttgga gatgagagat cttcctactg gaagcggcgg ttagtaggat gggcaagatc | 720 |
| tctcgcgtct tgacacacac acacacacac acaaatgagg tgggctgctc ctctttcctt | 780 |
| ccagaaggtc ggggttctgt tccacgaagc ccacagggaa ccttagggag ggcattcctc | 840 |
| cacagcggtg cctggacagc tttgtctgac ccaagccttg ctccggagct gactgcagag | 900 |
| actggaaagg gttagcagac aggaagcctg gctggggg | 938 |

Rat CD40 mRNA (GenBank accession no. AF 241231)

Rat CD40 mRNA sequence for targeting in accordance with the present invention is presented in SEQ ID NO:3. (See, Gao, Ph.D. thesis, Goettingen 2003).

| (SEQ ID NO:3): | |
|---|---|
| 1 | tgggacccct gtgatctggc tgctctgatc tcgctctgca atgctgcctt tgcctcagct |
| 61 | gtgcgcgctc tggggctgct tgttgacagc ggtccatcta ggacagtgtg ttacgtgcag |
| 121 | tgacaaacag tacctccaag gtggcgagtg ctgcgatttg tgccagccgg gaaaccgact |
| 181 | agttagccac tgcacagctc ttgagaagac ccaatgccaa ccgtgcgact caggcgaatt |
| 241 | ctcagctcac tggaacaggg agatccgctg ccaccagcac cgacactgcg aactcaatca |
| 301 | agggcttcag gttaagaagg agggcaccgc ggtntcagac actgtttgta cctgcaagga |
| 361 | agggcagcac tgcgccagca aggagtgcga gacgtgcgct cagcacaggc cctgtggccc |
| 421 | tggctttgga gtcgtgcaga tggccactga gactactgat accgtctgcc aaccctgccc |
| 481 | ggtcggattc ttctccaatg ggtcatcact ttttgaaaag tgtcatccat ggacaagctg |
| 541 | tgaagat |

Porcine CD40 cDNA

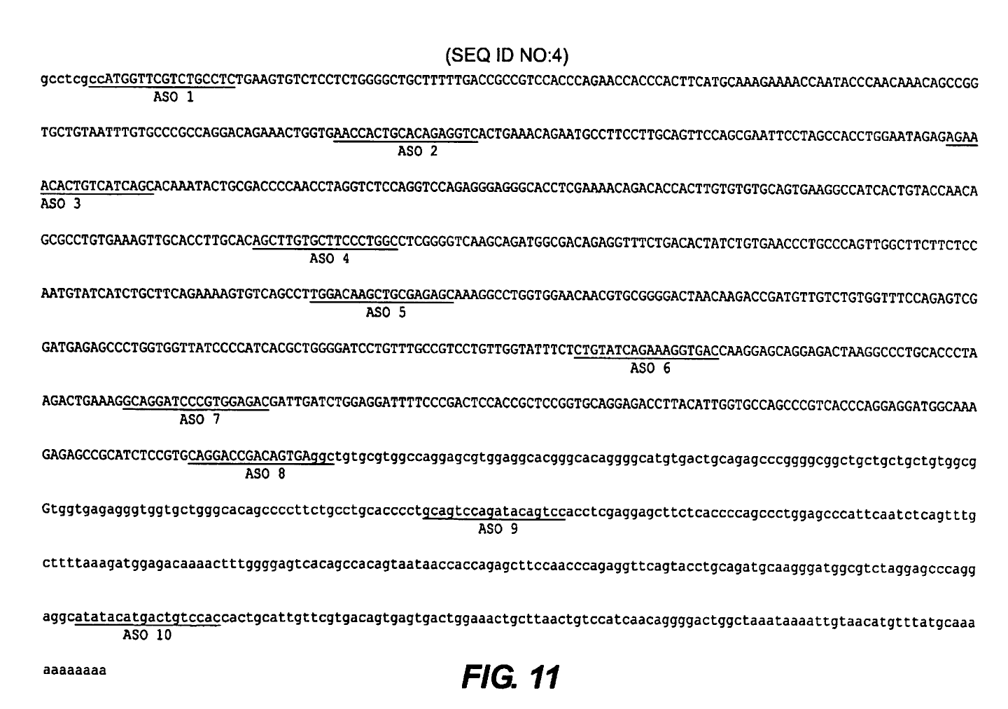

Porcine CD40 cDNA sequence for targeting in accordance with the present invention is presented in SEQ ID NO:4. (FIG. 11). Related sequence information is found in Rushworth, et al., Transplantation, 2002, 73(4), 635-642, the contents of which are incorporated by reference herein.

In addition, the following provide non-limiting examples of anti-CD40 oligonucleotides, e.g., antisense CD40 nucleic acid sequences, that are suitable for use in the present invention:

Oligonucleotides Against Human CD40

Examples of human antisense CD40 oligonucleotides are presented below. Further sequence information is found in published patent application number US 2004/0186071 and U.S. Pat. No. 6,197,584 to Bennett, et al., the contents of which are provided by reference herein. The SEQ ID NOs referred to by Bennett, et al. are provided to the right.

| | | | |
|---|---|---|---|
| SEQ ID NO: 5 | ccaggcggca ggaccact | Seq ID No: 1 | of Bennett et al. |
| SEQ ID NO: 6 | gaccaggcgg caggacca | Seq ID No.:2 | of Bennett et al. |
| SEQ ID NO: 7 | aggtgagacc aggcggca | Seq ID No: 3 | of Bennett et al. |
| SEQ ID NO: 8 | gcagaggcag acgaacca | Seq ID No: 5 | of Bennett et al. |
| SEQ ID NO: 9 | gcaagcagcc ccagagga | Seq ID No: 6 | of Bennett et al. |
| SEQ ID NO: 10 | ggtcagcaag cagcccca | Seq ID No.:7 | of Bennett et al. |
| SEQ ID NO: 11 | gacagcggtc agcaagca | Seq ID No: 8 | of Bennett et al. |
| SEQ ID NO: 12 | gatggacagc ggtcagca | Seq ID No: 9 | of Bennett et al. |
| SEQ ID NO: 13 | tctggatgga cagcggtc | Seq ID No.:10 | of Bennett et al. |
| SEQ ID NO: 14 | ggtggttctg gatggaca | Seq ID No: 11 | of Bennett et al. |
| SEQ ID NO: 15 | gtgggtggtt ctggatgg | Seq ID No: 12 | of Bennett et al. |
| SEQ ID NO: 16 | gcagtgggtg gttctgga | Seq ID No: 13 | of Bennett et al. |
| SEQ ID NO: 17 | ctggcacaaa gaacagca | Seq ID No: 15 | of Bennett et al. |
| SEQ ID NO: 18 | gtgcagtcac tcaccagt | Seq ID No: 20 | of Bennett et al. |
| SEQ ID NO: 19 | attccgtttc agtgaact | Seq ID No: 23 | of Bennett et al. |
| SEQ ID NO: 20 | ttcaccgcaa ggaaggca | Seq ID No: 25 | of Bennett et al. |
| SEQ ID NO: 21 | ctctgttcca ggtgtcta | Seq ID No: 26 | of Bennett et al. |
| SEQ ID NO: 22 | ctggtggcag tgtgtctc | Seq ID No: 27 | of Bennett et al. |
| SEQ ID NO: 23 | ggtgcccttc tgctggac | Seq ID No: 31 | of Bennett et al. |
| SEQ ID NO: 24 | ctgaggtgcc cttctgct | Seq ID No: 32 | of Bennett et al. |
| SEQ ID NO: 25 | gtgtctgttt ctgaggtg | Seq ID No: 33 | of Bennett et al. |
| SEQ ID NO: 26 | acaggtgcag atggtgtc | Seq ID No: 35 | of Bennett et al. |
| SEQ ID NO: 27 | gtgccagcct tcttcaca | Seq ID No: 37 | of Bennett et al. |
| SEQ ID NO: 28 | tgcaggacac agctctca | Seq ID No: 40 | of Bennett et al. |
| SEQ ID NO: 29 | gagcggtgca ggacacag | Seq ID No: 41 | of Bennett et al. |
| SEQ ID NO: 30 | aatctgcttg accccaaa | Seq ID No: 43 | of Bennett et al. |
| SEQ ID NO: 31 | gctcgcagat ggtatcag | Seq ID No: 46 | of Bennett et al. |
| SEQ ID NO: 32 | gcagggctcg cagatggt | Seq ID No: 47 | of Bennett et al. |
| SEQ ID NO: 33 | gactgggcag ggctcgca | Seq ID No: 49 | of Bennett et al. |
| SEQ ID NO: 34 | gcagatgaca cattggag | Seq ID No: 52 | of Bennett et al. |
| SEQ ID NO: 35 | tcgaaagcag atgacaca | Seq ID No: 53 | of Bennett et al. |
| SEQ ID NO: 36 | gtccaagggt gacatttt | Seq ID No: 54 | of Bennett et al. |
| SEQ ID NO: 37 | caggtctttg gtctcaca | Seq ID No: 57 | of Bennett et al. |
| SEQ ID NO: 38 | ctgttgcaca accaggtc | Seq ID No: 58 | of Bennett et al. |
| SEQ ID NO: 39 | gtttgtgcct gcctgttg | Seq ID No: 59 | of Bennett et al. |
| SEQ ID NO: 40 | gtcttgtttg tgcctgcc | Seq ID No: 60 | of Bennett et al. |
| SEQ ID NO: 41 | caccaccagg gctctcag | Seq ID No: 64 | of Bennett et al. |
| SEQ ID NO: 42 | gggatcacca ccagggct | Seq ID No: 65 | of Bennett et al. |
| SEQ ID NO: 43 | gtcgggaaaa ttgatctc | Seq ID No: 71 | of Bennett et al. |
| SEQ ID NO: 44 | ggagccagga agatcgtc | Seq ID No: 73 | of Bennett et al. |
| SEQ ID NO: 45 | tggagccagg aagatcgt | Seq ID No: 74 | of Bennett et al. |
| SEQ ID NO: 46 | tggcatccat gtaaagtc | Seq ID No: 77 | of Bennett et al. |
| SEQ ID NO: 47 | ggtgcagcct cactgtct | Seq ID No: 81 | of Bennett et al. |
| SEQ ID NO: 48 | aactgcctgt ttgcccac | Seq ID No: 82 | of Bennett et al. |

The following siRNA sequences are suitable for use in the present invention. (See, e.g., Pluvinet, et al., Blood, 2004, 104(12), 3642-3646), the contents of which are incorporated by reference herein.

| | |
|---|---|
| (SEQ ID NO:49): | |
| 5_-GCGAAUUCCUAGACACCUGUU-3 | (siRNA-2 of |
| 3_-UUCGCUUAAGGAUCUGUGGAC-5_ | Pluvinet et al.) |
| (SEQ ID NO:50): | |
| 5_-CUGGUGAGUGACUGGACAGUU-3 | (siRNA-6 of |
| 3_-UUGACCACUGACUGACGUGUC-5_ | Pluvinet et al.) |
| SEQ ID NO:51): | |
| 5_-UACUGCGACCGCAACCUAGUU-3 | (siRNA-8 of |
| 3_-UUAUGACGCUGGGGUUGGAUC-5_ | Pluvinet et al.) |

All siRNA contain a 2 nucleotide overhang at 3′ends.

Oligonucleotides Against Murine CD40

Examples of murine antisense CD40 oligonucleotides are presented below. Further sequence information is found in published patent application number US 2004/0186071 to Bennett, et al., the contents of which are hereby incorporated by reference herein. The SEQ ID NOs referred to by Bennett, et al. are provided to the right.

Murine

| | | | |
|---|---|---|---|
| SEQ ID NO: 52 | agacaccatc gcag | Seq. ID No. 116 | of Bennett et al. |
| SEQ ID NO: 53 | gcgagatcag aagag | Seq. ID No. 117 | of Bennett et al. |
| SEQ ID NO: 54 | cgctgtcaac aagca | Seq. ID No. 118 | of Bennett et al. |
| SEQ ID NO: 55 | ctgccctaga tggac | Seq. ID No. 119 | of Bennett et al. |
| SEQ ID NO: 56 | ctggctggca caaat | Seq. ID No. 120 | of Bennett et al. |
| SEQ ID NO: 57 | cttgtccagg gataa | Seq. ID No. 123 | of Bennett et al. |
| SEQ ID NO: 58 | cacagatgac attag | Seq. ID No. 124 | of Bennett et al. |
| SEQ ID NO: 59 | tgatatagag aaaca | Seq. ID No. 125 | of Bennett et al. |
| SEQ ID NO: 60 | ctcattatcc tttgg | Seq. ID No. 127 | of Bennett et al. |
| SEQ ID NO: 61 | ggttcagacc agg | Seq. ID No. 128 | of Bennett et al. |
| SEQ ID NO: 62 | tttatttagc cagta | Seq. ID No. 130 | of Bennett et al. |
| SEQ ID NO: 63 | agccccacgc actgg | Seq. ID No. 131 | of Bennett et al. |
| SEQ ID NO: 64 | tctcactcct atcccagt | Seq. ID No. 134 | of Bennett et al. |
| SEQ ID NO: 65 | attagtctga ctcgt | Seq. ID No. 138 | of Bennett et al. |
| SEQ ID NO: 66 | acattagtct gacte | Seq. ID No. 139 | of Bennett et al. |
| SEQ ID NO: 67 | cagatgacat tagtc | Seq. ID No. 142 | of Bennett et al. |
| SEQ ID NO: 68 | ctggactcac cacag | Seq. ID No. 143 | of Bennett et al. |
| SEQ ID NO: 69 | ggactcacca cagat | Seq. ID No. 144 | of Bennett et al. |
| SEQ ID NO: 70 | actcaccaca gatga | Seq. ID No. 145 | of Bennett et al. |
| SEQ ID NO: 71 | teaccacaga tgaca | Seq. ID No. 146 | of Bennett et al. |
| SEQ ID NO: 72 | accacagatg acatt | Seq. ID No. 147 | of Bennett et al. |
| SEQ ID NO: 73 | agatgacatt ag | Seq. ID No. 153 | of Bennett et al. |
| SEQ ID NO: 74 | cagatgacat tag | Seq. ID No. 154 | of Bennett et al. |
| SEQ ID NO: 75 | acagatgaca ttag | Seq. ID No. 155 | of Bennett et al. |
| SEQ ID NO: 76 | ccacagatga cattag | Seq. ID No. 156 | of Bennett et al. |
| SEQ ID NO: 77 | accacagatg acattag | Seq. ID No. 157 | of Bennett et al. |
| SEQ ID NO: 78 | caccacagat gacattag | Seq. ID No. 158 | of Bennett et al. |
| SEQ ID NO: 79 | tcaccacaga tgacattag | Seq. ID No. 159 | of Bennett et al. |
| SEQ ID NO: 80 | ctcaccacag atgacattag | Seq. ID No. 160 | of Bennett et al. |

Oligonucleotides Against Rat CD40

Examples of rat antisense CD40 oligonucleotides are presented below. (See, Gao, Ph.D. thesis, 2003, University of Göttingen, Germany).

SEQ ID NO:81

accgctgtcaacaagcagc (rAS2 of Gao)

SEQ ID NO:82

tcctagatggaccgctgt (rAS3 of Gao)

SEQ ID NO:83

taacacactgtcctag (rAS4 of Gao)

Oligonucleotides Against Porcine CD40

Examples of porcine antisense CD40 oligonucleotides are presented below. See, Rushworth, et al., Transplantation, 2002, 73(4), 635-642, the contents of which are incorporated by reference herein.

SEQ ID NO:84

gctgatgacagtgtttct (Aso3 of Rushworth et al.)

SEQ ID NO:85

gcctcactctcgctcctg (Aso8 of Rushworth et al.)

SEQ ID NO:86

ggactgtatctggactgc (Aso9 of Rushworth et al.)

SEQ ID NO:87

gtggacagtcatgtatat (Aso10 of Rushworth et al.)

### GLOSSARY OF COMMON ABBREVIATED LIPID NAMES

DMPC Dimyristoylphosphatidylcholine
DPPC Dipalmitoylphosphatidylcholine
DSPC Distearoylphosphatidylcholine
POPC Palmitoyl-oleoylphosphatidylcholine
DOPC Dioleoylphosphatidylcholine
DOPE Dioleoylphosphatidylethanolamine
DMPE Dimyristoylphosphatidylethanolamine
DPPE Dipalmitoylphosphatidylethanolamine
DOPG Dioleoylphosphatidylglycerol
POPG Palmitoyl-oleoylphosphatidylglycerol
DMPG Dimyristoylphosphatidylglycerol
DPPG Dipalmitoylphosphatidylglycerol
DMPS Dimyristoylphosphatidylserine
DPPS Dipalmitoylphosphatidylserine
DOPS Dioleoylphosphatidylserine
POPS Palmitoyl-oleoylphosphatidylserine
DMPA Dimyristoylphosphatidic acid
DPPA Dipalmitoylphosphatidic acid
DOPA Dioleoylphosphatidic acid
POPA Palmitoyl-oleoylphosphatidic acid
CHEMS Cholesterolhemisuccinate
DC-Chol 3-β-[N-(N′,N′-dimethylethane) carbamoyl] cholesterol
CetylP Cetylphosphate
DODAP (1,2)-dioleoyloxypropyl)-N,N-dimethylammonium chloride
DOEPC 1,2-dioleoyl-sn-glycero-3-ethylphosphocholine
DAC-Chol 3-β-[N-(N,N′-dimethylethane) carbamoyl]cholesterol
TC-Chol 3-β-[N-(N′,N′, N′-trimethylaminoethane) carbamoyl] cholesterol
DOTMA (1,2-dioleyloxypropyl)-N,N,N-trimethylammoniumchlorid) (Lipofectin®)
DOGS ((C18)₂GlySper3⁺) N,N-dioctadecylamido-glycyl-spermine (Transfectam®)
CTAB Cetyl-trimethylammoniumbromide,
CPyC Cetyl-pyridiniumchloride
DOTAP (1,2-dioleoyloxypropyl)-N,N,N-trimethylammonium salt
DMTAP (1,2-dimyristoyloxypropyl)-N,N,N-trimethylammonium salt
DPTAP (1,2-dipalmitoyloxypropyl)-N,N,N-trimethylammonium salt
DOTMA (1,2-dioleyloxypropyl)-N,N,N-trimethylammonium chloride)
DORIE (1,2-dioleyloxypropyl)-3 dimethylhydroxyethyl ammoniumbromide)
DDAB Dimethyldioctadecylammonium bromide
DPIM 4-(2,3-bis-palmitoyloxy-propyl)-1-methyl-1H-imidazole
CHIM Histaminyl-Cholesterolcarbamate
MoChol 4-(2-Aminoethyl)-Morpholino-Cholesterolhemisuccinate
HisChol Histaminyl-Cholesterolhemisuccinate.
HCChol Nα-Histidinyl-Cholesterolcarbamate
HistChol Nα-Histidinyl-Cholesterol-hemisuccinate.
AC Acylcarnosine, Stearyl- & Palmitoylcarnosine
HistDG 1,2-Dipalmitoylglycerol-hemisuccinate-Nα-Histidinyl-hemisuccinate, & Distearoyl-,Dimyristoyl, Dioleoyl or palmitoyl-oleoylderivatives
IsoHistSuccDG 1,2-Dipalmitoylglycerol-Oα-Histidinyl-Nα-hemisuccinat, & Distearoyl-, Dimyristoyl, Dioleoyl or palmitoyl-oleoylderivatives
DGSucc 1,2-Dipalmitoyglycerol-3-hemisuccinate & Distearoyl-, dimyristoyl-Dioleoyl or palmitoyl-oleoylderivatives

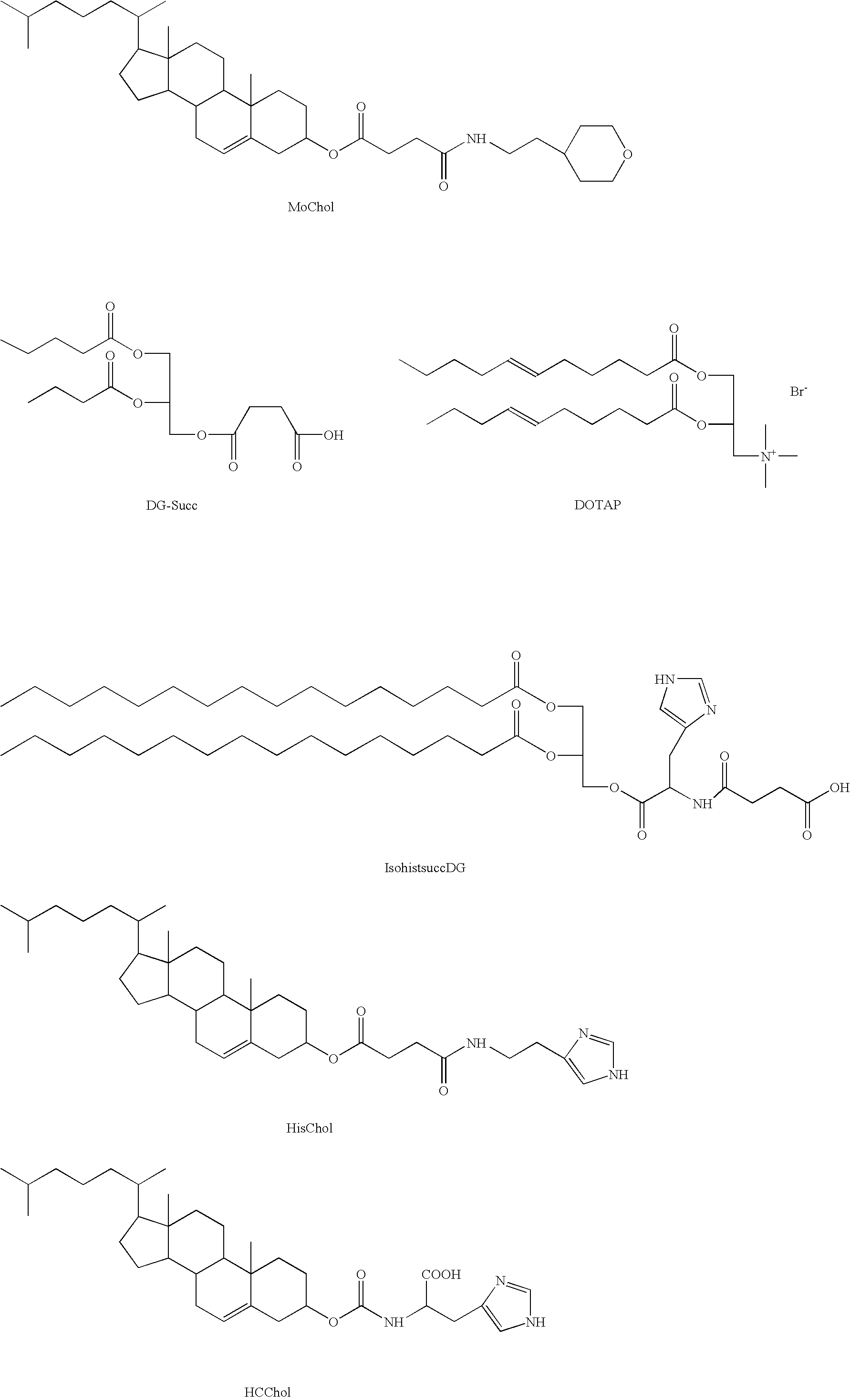

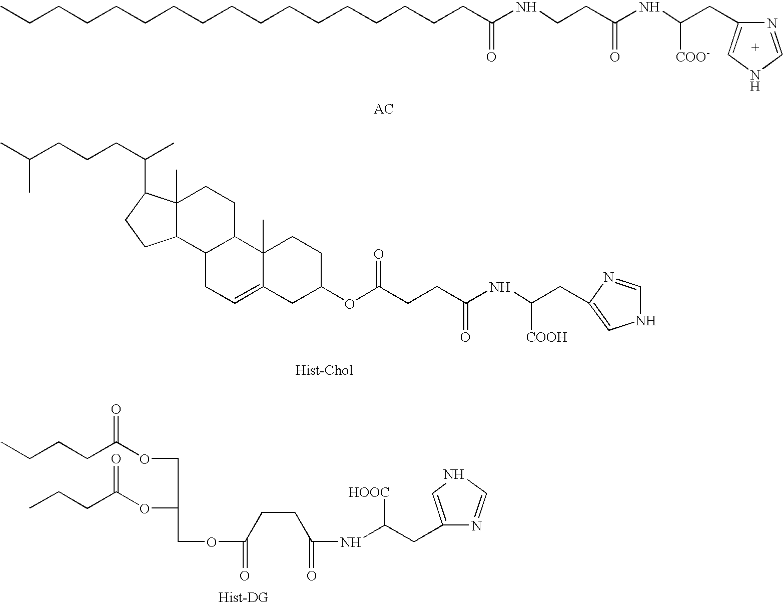

What is claimed is:

1. A pharmaceutical composition comprising an oligonucleotide as an active agent, said oligonucleotide adapted to target nucleic acids encoding CD40 so as to modulate the expression of CD40 in mammalian cells, and an amphoteric liposome as an excipient.

2. The pharmaceutical composition according to claim 1, wherein said liposome has an isoelectric point of between 4 and 7.4.

3. The pharmaceutical composition according to claim 1, wherein said amphoteric liposome is negatively charged or neutral at pH 7.4 and cationic at pH 4.

4. The pharmaceutical composition according to claim 1, wherein said amphoteric liposome is formed from a lipid phase comprising an amphoteric lipid.

5. The pharmaceutical composition according to claim 4, wherein said lipid phase comprises 5 to 30 mol. % of said amphoteric lipid.

6. The pharmaceutical composition according to claim 4, wherein said amphoteric lipid is selected from the group consisting of HistChol, HistDG, isoHistSuccDG, Acylcarnosin and HCCHol.

7. The pharmaceutical composition according to claim 1, wherein said amphoteric liposome is formed from a lipid phase comprising a mixture of lipid components with amphoteric properties.

8. The pharmaceutical composition according to claim 7, wherein said mixture of lipid components comprises anionic or cationic components and wherein at least one of the components is pH responsive.

9. The pharmaceutical composition according to claim 8, wherein said mixture of lipid components comprises (i) a stable cationic lipid and a chargeable anionic lipid, (ii) a chargeable cationic lipid and chargeable anionic lipid or (iii) a stable anionic lipid and a chargeable cationic lipid.

10. The pharmaceutical composition according to claim 9, wherein said lipid components comprise one or more anionic lipids selected from the group consisting of DGSucc, DMPS, DPPS, DOPS, POPS, DMPG, DPPG, DOPG, POPG, DMPA, DPPA, DOPA, POPA, CHEMS and Cetyl-P.

11. The pharmaceutical composition according to claim 9, wherein said lipid components comprise one or more anionic lipids selected from the group consisting of DGSucc, DOPA, CHEMS and Cetyl-P.

12. The pharmaceutical composition according to claim 8, wherein said lipid components comprise one or more cationic lipids selected from the group consisting of DMTAP, DPTAP, DOTAP,DC-Chol, MoChol, HisChol, DPIM, CHIM, DORIE, DDAB,DAC-Chol, TC-Chol, DOTMA, DOGS, (C18)₂Gly⁺N,N-dioctadecylamido-glycin, CTAP, CPyC, DODAP and DOEPC.

13. The pharmaceutical composition according to claim 8, wherein said lipid components comprise one or more cationic lipids selected from the group consisting of DOTAP, DC-Chol, MoChol and HisChol.

14. The pharmaceutical composition according to claim 4, wherein said lipid phase further comprises a neutral phospholipid.

15. The pharmaceutical composition according to 14, wherein said lipid phase comprises a neutral phosphatidylcholine.

16. The pharmaceutical composition according to claim 15, wherein said phosphatidylcholine is selected from the group consisting of DMPC, DPPC, DSPC, POPC, DOPC, natural source phosphatidylcholines, soy bean PC and egg PC.

17. The pharmaceutical composition according to claim 15, wherein said neutral phosphatidylcholine is selected from the group consisting of POPC, natural or hydrogenated soy bean PC, natural or hydrogenated egg PC, DMPC, DPPC and DOPC.

18. The pharmaceutical composition according to claim 15, wherein said phosphatidylcholine comprises POPC, non-hydrogenated soy bean PC, or non-hydrogenated egg PC.

19. The pharmaceutical composition according to claim 15, wherein said lipid phase comprises at least 15 mol. % of said phosphatidylcholine.

20. The pharmaceutical composition according to claim 19, wherein said lipid phase comprises about 60 mol. % POPC, about 10 mol. % DOTAP and about 30 mol. % CHEMS.

21. The pharmaceutical composition according to claim 14, wherein said neutral lipid comprises a phosphatidylethanolamine.

22. The pharmaceutical composition according to claim 21, wherein said phosphatidylethanolamine is selected from DOPE, DMPE, or DPPE.

23. The pharmaceutical composition according to claim 21, wherein said lipid phase comprises at least 20 mol. % of phosphatidylcholine and phosphatidylethanolamine.

24. The pharmaceutical composition according to claim 21, wherein said lipid phase comprises a mixture of anionic and cationic lipids with amphoteric properties, phosphatidylcholine and phosphatidylethanolamine.

25. The pharmaceutical composition according to claim 24, wherein said cationic lipid comprises MoChol and said anionic lipid comprises CHEMS or DMG-Succ.

26. The pharmaceutical composition according to claim 25, wherein said lipid phase comprises:
(a) about 15 mol. % POPC, about 45 mol. % DOPE, about 20 mol. % MoChol and about 20 mol. % CHEMS;
(b) about 10 mol. % POPC, about 30 mol. % DOPE, about 30 mol. % MoChol and about 30 mol. % CHEMS;
(c) about 10 mol. % POPC, about 30 mol. % DOPE, about 20 mol. % MoChol and about 40 mol. % CHEMS; or
(d) about 6 mol. % POPC, about 24 mol. % DOPE, about 47 mol. % MoChol and about 23 mol. % CHEMS.

27. The pharmaceutical composition according to claim 21, wherein said lipid phase comprises DOPE and POPC.

28. The pharmaceutical composition according to claim 14, wherein said lipid phase further comprises cholesterol.

29. The pharmaceutical composition according to claim 28, wherein said lipid phase comprises from 30 mol. % to 50 mol. % cholesterol.

30. The pharmaceutical composition according to claim 28, wherein said lipid phase comprises about 30 mol. % POPC, about 10 mol. % DOTAP, about 20 mol. % CHEMS and about 40 mol. % Chol.

31. The pharmaceutical composition according to claim 28, wherein said lipid phase comprises about 60 mol. % POPC, about 20 mol. % HistChol and about 20 mol. % Chol.

32. The pharmaceutical composition according to claim 23, wherein said composition further comprises a vehicle and is formulated for systemic delivery.

33. The pharmaceutical composition according to claim 1, wherein said composition further comprises a vehicle and is formulated for local administration.

34. The pharmaceutical composition according to claim 1, wherein said liposome has a size in the range 50 to 500 nm.

35. The pharmaceutical composition according to claim 1, wherein said oligonucleotide is an antisense oligonucleotide of 15 to 50 basepairs in length.

36. The pharmaceutical composition according to claim 35, wherein said oligonucleotide contains phosphothioate linkages, 2′MOE modified nucleobases, LNA nucleobases, FANA nucleobases, naturally occurring ribonucleotides, or naturally occurring deoxyribonucleotides.

37. The pharmaceutical composition according to claim 1, wherein said oligonucleotide comprises a siRNA of 15 to 50 basepairs in length.

38. The pharmaceutical composition according to claim 1, wherein said oligonucleotide targets the human CD40 gene.

39. A method of treating or preventing an inflammatory, immune or autoimmune disorder, comprising administering to a recipient in need thereof, the pharmaceutical composition according to claim 1 in an amount effective to treat or prevent the condition.

40. A method of treating or preventing a disease or condition selected from graft rejection, graft-versus-host disease, multiple sclerosis, systemic lupus erythematosous, rheumatoid arthritis, asthma, inflammatory bowel disease, psoriasis, or thyroiditis, comprising administering to a recipient in need thereof, the pharmaceutical composition according to claim 32 in an amount effective to treat or prevent the disease or condition.

41. A method of treating or preventing a disease or condition selected from graft rejection, graft-versus-host disease, inflammatory bowel disease, Morbus Crohn, or Colitis ulcerosa, comprising administering to a recipient in need thereof, the pharmaceutical composition according to claim 33 in an amount effective to treat or prevent the disease or condition.

42. The method according to claim 39, wherein the recipient is a human or non-human animal.

43. The method according to claim 40, wherein the recipient is a human or non-human animal.

44. The method according to claim 41, wherein the recipient is a human or non-human animal.
